(19) United States
(12) Patent Application Publication
(10) Pub. No.: US 2006/0211112 A1
(43) Pub. Date: Sep. 21, 2006

(54) Growth of human dendritic cells for cancer immunotherapy in closed system using microcarrier beads

(76) Inventors: Paul E. Harris, New York, NY (US); Charles Hesdorffer, Bronx, NY (US)

Correspondence Address: John P White;Cooper & Dunham LLP, 1185 Avenue of the Americas, New York, NY, 10036, US

(21) Appl. No.: 10/433,387

(22) PCT Filed: Nov. 30, 2001
(86) PCT No.: PCT/US01/45099

§ 371 Date: May 10, 2004

Related U.S. Application Data
(63) Continuation-in-part of application No. 09/726,883, filed on Nov. 30, 2000, now abandoned.

(51) Int. Cl. C12N 5/08; C12M 3/00

(52) U.S. Cl. 435/372; 435/289.1

(57) ABSTRACT

A method and apparatus for reproducibly generating dendritic cells are provided. Blood mononuclear cells are loaded into a cell culture container containing microcarrier beads therein. Tissue culture comprising the cells loaded in the container is incubated for a predetermined period. Nonadherent cells and cells adhered to the beads are separated. Dendritic cell culture medium may be prepared and transferred to the container after the cells which adhere to the beads are separated from the nonadherent cells. The tissue culture incubated for the predetermined time period may be washed to remove nonadherent cells. The beads may be allowed to settle and supernatant is expressed off. The container may comprise a gas permeable cell culture bag.

This application is a continuation-in-part and claims priority of U.S. Ser. No. 09/726,883, filed Nov. 30, 2000, the contents of which are hereby incorporated by reference.

### BACKGROUND

The present application relates to a method of growing adherence-dependent hematopoietic cells. In particular, dendritic cells are grown in a closed system using microcarrier beads.

Throughout this application, various publications are referenced by author and date. Full citations for these publications may be found listed alphabetically at the end of the specification immediately preceding the claims. The disclosures of these publications in their entireties are hereby incorporated by reference into this application in order to more fully describe the state of the art as known to those skilled therein as of the date of the invention described and claimed herein.

Dendritic cells (DCs) constitute potent antigen-presenting cells. They may be derived from bone marrow progenitor cells and circulate in small numbers in the peripheral blood. As antigen-presenting cells, DCs are able to induce activation of T-cells with a high degree of efficiency. They are highly specialized and optimally equipped for their task, since dendritic cells express molecules which are required for presenting antigen in large quantity. Important adhesion molecules, which guarantee intimate contact with the target cell, are present on the surface of the dendritic cells.

Due to low frequency of DC in peripheral blood, ex vivo expansion and maturation of DC precursors are required for their clinical application (Bartholeyns et al., 1998).

There is a need to refine DC culture methods for clinical use in immunotherapy for cancer patients. Most DC culture systems are initiated from the adherent fraction of peripheral blood mononuclear cells, selected using open polystyrene flasks, followed by washing and then culture in serum-free medium containing GM-CSF and IL-4 or IL-7 (as well as other maturational cytokines) {Schuler et al., 1997; Di Nicola et al., 1998}. The open system is labor intensive and poses an increased risk of microbial contamination to the expanded product, the patient and the technician.

An alternative to the open flask is a closed system for culturing populations of monocyte enriched peripheral blood mononuclear cells using flexible gas permeable cell culture bags and sterile connecting devices (Glaser et al., 1999). Growing human DC in plastic bags, even under clinical grade and using good manufacturing practices, have poor yields because the surface of the bags is suboptimal.

### SUMMARY

The application provides a method of reproducibly generating dendritic cells, comprising the steps of:
(a) loading blood mononuclear cells into a cell culture container containing microcarrier beads therein;
(b) incubating for a predetermined time period tissue culture comprising the cells loaded in the container in step (a); and
(c) separating nonadherent cells and cells adhered to the beads.

The application also provides a method of reproducibly generating dendritic cells, comprising the steps of:
(a) loading microcarrier beads into a cell culture container;
(b) loading blood mononuclear cells into the container;
(c) incubating for a predetermined time period tissue culture comprising the mononuclear cells loaded in the container in step (b); and
(d) separating nonadherent cells and cells adhered to the beads.

The application also provides an apparatus for reproducibly generating dendritic cells, comprising:
a cell culture container; and
a plurality of microcarrier beads contained within the cell culture container.

The container may comprise a gas permeable cell culture bag. The container is a closed vessel.

The microcarrier beads may comprise styrene copolymer beads. The microcarrier beads may comprise polystyrene copolymer beads.

A ratio of a combined surface area of the microcarrier beads and the container to a volume of the container volume preferably is a value that allows the container to hold enough media for a predetermined time period of incubation.

### BRIEF DESCRIPTION OF THE DRAWINGS

The present application would be more readily understood from the following detailed description by referring to the accompanying drawings wherein:

FIG. 1 shows a block diagram of an apparatus for reproducibly generating dendritic cells, in accordance with an embodiment of the present application;

FIG. 2A shows a flow chart of a method of reproducibly generating dendritic cells, in accordance with an embodiment of the present application;

FIG. 2B shows a flow chart of a method of reproducibly generating dendritic cells, in accordance with another embodiment of the present application;

FIG. 3 shows a flow chart of a method, in accordance with another embodiment, for reproducibly generating dendritic cells;

FIG. 4 shows a table comparing dendritic cell culture methods;

FIG. 5 shows a table showing typical immunophenotype of cultured dendritic cells;

FIGS. 6A through 6D show a manufacturing flow sheet of a method, in accordance with another embodiment of the present application, for reproducibly generating dendritic cells;

FIGS. 7A-7F show histograms corresponding to experimental data obtained through immunofluorescent flow cytometry, depicting the number of cells exhibiting various fluorescence intensities;

FIG. 8 shows a XY-scatterplot analysis of log base 2-transformed expression data;

FIG. 9-13 show dendrograms corresponding to data measured by using a cDNA array with elements representing a plurality of distinct human genes;

FIGS. 14A and 14B show patterns of expression using semi-quantitative reverse transcription polymerase chain reaction of four genes (CD37, CD81, CD53 and BCL-6);

FIG. 15 shows a table corresponding to gene expression changes in cultured adherent cells treated with GM-CSF and IL-4;

FIG. 16 shows a table corresponding to gene expression changes in cultured adherent cells treated with GM-CSF and IL-7;

FIG. 17 shows a table corresponding to gene expression changes in immature DCs treated with IFN γ;

FIG. 18 shows a table corresponding to gene expression changes in immature DCs treated with TNF α; and

FIG. 19 shows a table corresponding to gene expression changes in immature DCs treated with s CD40 L trimer.

### DETAILED DESCRIPTION

The present disclosure provides a novel and unobvious tool for reproducible generation of dendritic cells. Addition of selected, sterile plastic microcarrier beads enhances production of human dendritic cells (DC) in gas permeable cell culture bags. The method also may be adapted for growth of other adherence-dependent hematopoietic cells.

The present application, in accordance with an embodiment, provides an apparatus for reproducibly generating dendritic cells comprising a cell culture container and a plurality of microcarrier beads contained within the cell culture container.

The present application, in accordance with an embodiment, provides a method of reproducibly generating dendritic cells, comprising the steps of (a) loading blood mononuclear cells into a cell culture container containing microcarrier beads therein, (b) incubating for a predetermined time period tissue culture comprising the cells loaded in the container in step (a), and (c) separating nonadherent cells and cells adhered to the beads.

The present application, in accordance with another embodiment, provides a method of reproducibly generating dendritic cells, comprising the steps of (a) loading microcarrier beads into a cell culture container, (b) loading blood mononuclear cells into the container, (c) incubating for a predetermined time period tissue culture comprising the mononuclear cells loaded in the container in step (b), and (d) separating nonadherent cells and cells adhered to the beads.

The container may comprise a gas permeable cell culture bag. The container is a closed vessel.

The microcarrier beads may comprise styrene copolymer beads and/or polystyrene copolymer beads.

The tissue culture incubated for the predetermined time period may be washed to remove nonadherent cells. After the tissue culture is incubated for a predetermined time period, the beads may be allowed to settle and supernatant expressed off.

The method further may comprise (d) preparing dendritic cell culture medium, and (e) transferring the dendritic cell culture medium to the container after the nonadherent cells and the cells adhered to the beads are separated. The method further also may comprise (f) incubating the container for a second predetermined time period after step (e), (g) agitating contents of the container incubated in step (f), and (h) harvesting cell culture suspension by expression into transfer bags using a sterile connecting device after the beads agitated in step (g) are allowed to settle.

Samples may be removed from the container for quality control after the nonadherent cells and the cells adhered to the beads are separated. The quality control may include at least one of viability staining, microbial analysis, cell enumeration, microscopic examination of dendritic cell morphology, and immunophenotyping to determine a purity of the dendritic cell preparation.

The blood mononuclear cells may be obtained by apheresis.

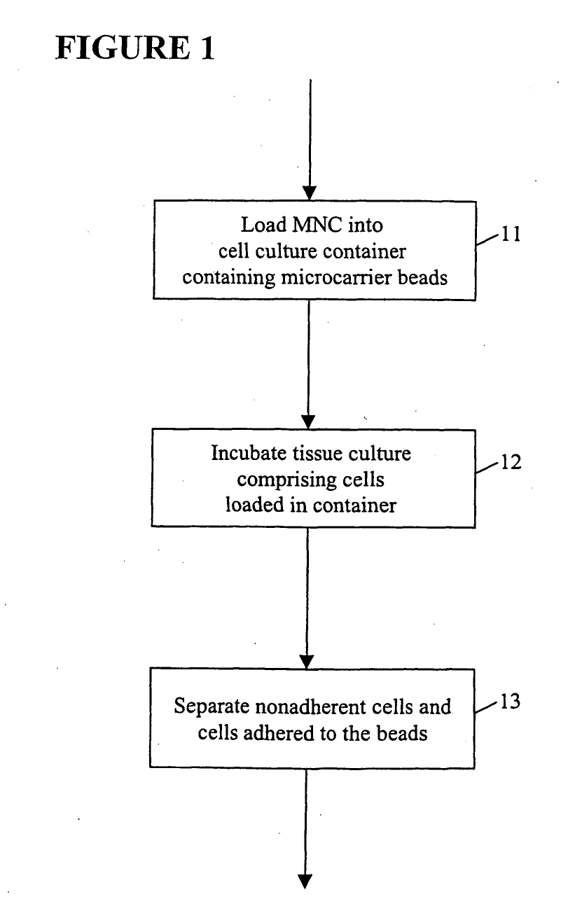

An apparatus for reproducibly generating dendritic cells, in accordance with an embodiment, will be described with reference to FIG. 1. Apparatus 1 includes a cell culture container 3 and a plurality of microcarrier beads 5. The container 3 may comprise a gas permeable cell culture bag. The container 3 is a closed vessel. The microcarrier beads 5 may comprise styrene copolymer beads and/or polystyrene copolymer beads.

The apparatus 1 also may be provided with a tubing harness including connectors 7a and 7b coupled to respective ports in the container 3 which facilitate the loading of cells into the container via a transfer process which is preferably substantially sterile, while maintaining the close environment provided by the container. Loading of the container may be manual or via a transfer pump. To optimize the sterility of the apparatus when loading is not being performed, a cap may be provided.

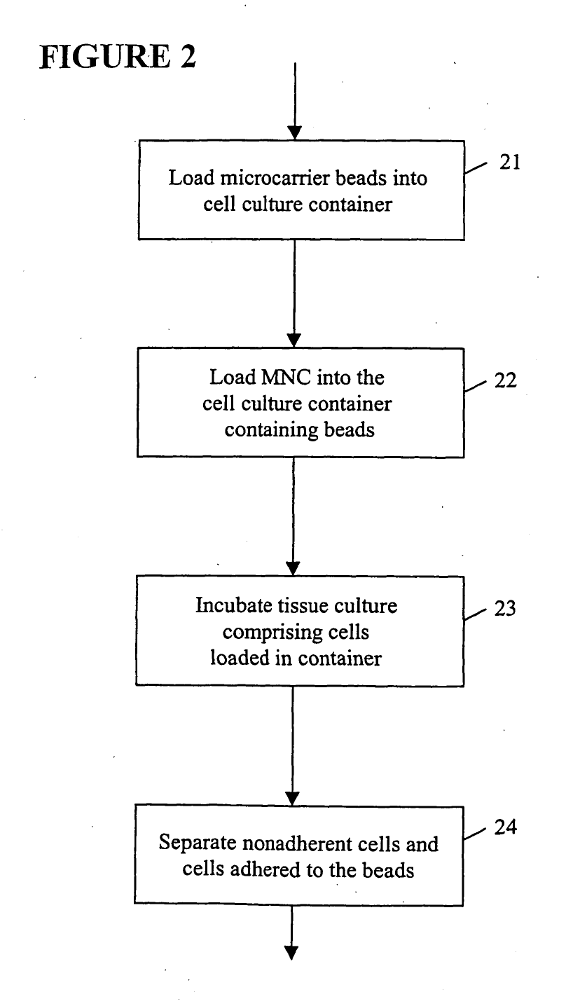

A method of reproducibly generating dendritic cells, in accordance with one embodiment of the present application, will be described with reference to FIGS. 1 and 2A. Blood mononuclear cells are loaded into the cell culture container 3 containing the microcarrier beads 5 (step 11). Tissue culture comprising the cells loaded in the container 3 are incubated for a predetermined period (step 12). Nonadherent cells and cells adhered to the beads 5 are separated (step 13).

A method of reproducibly generating dendritic cells, in accordance with another embodiment of the present application (FIG. 2B), includes first loading the microcarrier beads 5 into the cell culture container 3 (step 21), for example through a valve (7a or 7b) provided in the cell culture container. Blood mononuclear cells then are loaded into the container 3 containing the microcarrier beads 5 (step 22). Tissue culture comprising the cells loaded in the container 3 are incubated for a predetermined period (step 23). Nonadherent cells and cells adhered to the beads 5 are separated (step 24).

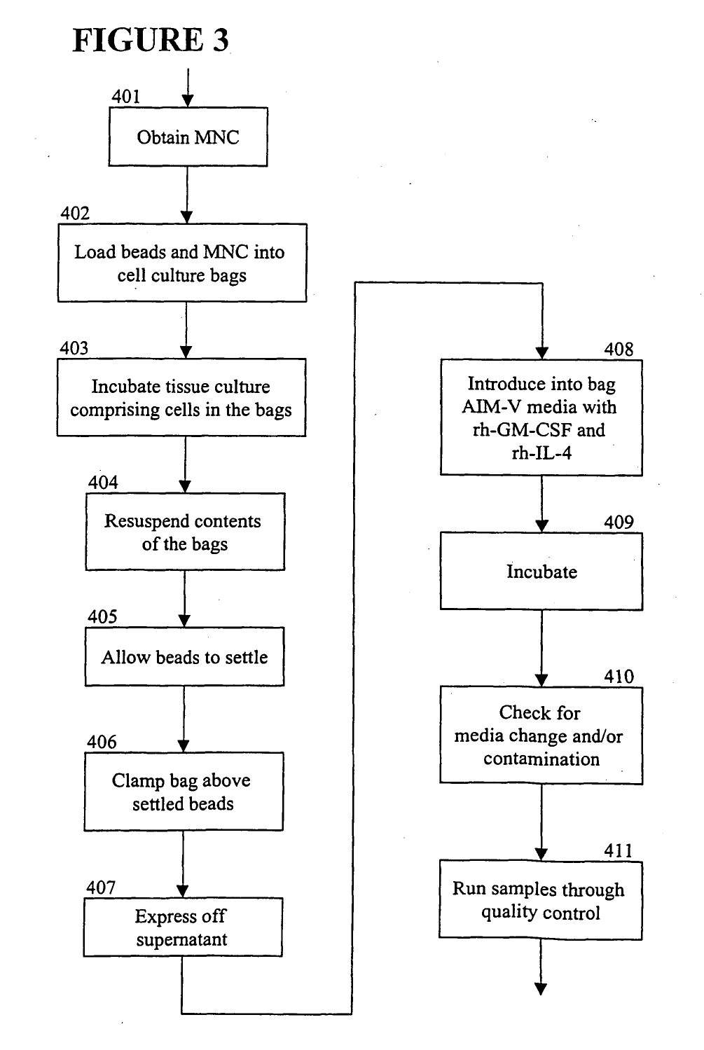

The subject matter of the present application is illustrated in the Experimental Details section which follows with reference to FIGS. 3 through 6D. These sections are set forth to aid in an understanding of the application but are not intended to, and should not be construed to, limit in any way the application as set forth in the claims which follow thereafter.

### EXPERIMENTAL DETAILS

### Example 1

One desirable property of blood mononuclear cell (MNC) products suitable for DC culture is collection of a maximum number of monocytes and monocyte precursors with a minimum number of red blood cells, lymphocytes and platelets. This may be accomplished by pheresing donors on an apheresis system (e.g., Spectra, COBE BCT, Lakewood, Colo.) using a mononuclear cell program.

Thus, the MNC for the DC culture may be obtained (step 401) by apheresis, under informed consent, from G-CSF mobilized donors, in accordance with one embodiment. Donors may undergo, for example, a 10-liter apheresis. The collection schema may utilize a separation fraction of 250, equivalent to a velocity of 635 rpm at an inlet flow of 50 ml per min. Materials and reagents used for the apheresis and DC culture preferably are sterile and/or endotoxin free and FDA approved for human use.

Without use of microcarrier beads, the yield of DCs per unit of culture surface area in closed gas permeable cell culture bags is less than the yield in open flask systems. To improve the yields of DCs in a closed system, styrene copolymer beads (e.g., 90-500 micron diameter, density≧1.04 g/cm³, SoloHill Engineering, Inc., Ann Arbor, Mich.) are introduced into the bags, in accordance with one embodiment of the present application, to increase the available surface area (e.g., by 380 cm²) and supply a surface area similar to that found in the flasks {see, e.g., M. Kiremitci et al., Cell adhesion to the surfaces of polymeric beads, 18 Biomater. Artif. Cells Artif. Organs 599-603 (1990)}. Also, the beads have a density that allows them to sink/settle in due course.

In accordance with one (FIGS. 3 through 5) of many possible embodiments, 1 gram of gamma radiation sterilized beads and 10×10*8 total cells/bag of MNC product are diluted in 100 mls AIM-V (e.g., from GIBCO, Grand Island, N.Y.) and loaded into gas permeable cell culture bags (e.g., Lifecell X-fold Cell Culture Containers PL2417, 180 cm², Nexell Therapeutics, Irvine, Calif.), under a biological safety cabinet (step 402).

The tissue culture bags then are incubated (step 403), for example, in a humidified 37° C., 5% CO₂ atmosphere for approximately four hours. After four hours the contents of the bag are gently resuspended (step 404), the beads are allowed to settle for 5 minutes at 1×g (step 405), and the bag is clamped 1 cm above the settled beads (step 406). The supernatant then is expressed off (step 407) using a transfer bag and a sterile connecting device (e.g, from Terumo Corp., Phoenix, Ariz.). This procedure (steps 402-407) may be repeated three times with 50 mls AIM-V media. As control for adherence, a sample of the expressed cells may be immunophenotyped for monocyte markers (e.g., CD14 and CD11c). Adherence of MNC to the bag and bead surface may be inferred by a decrease in the percent of CD14 and CD11c positive cells in the expressed fraction relative to the apheresis product.

After removal of nonadherent cells, 100 ml of AIM-V media containing rh-GM-CSF (e.g., 25 ng/ml, Sargramostim, Immunex, Seattle, Wash.) and rh IL-4 (e.g., 1000 U/ml, Sigma, St. Louis, Mo.) is introduced into the tissue culture bags (step 408). The bags may be placed into a dedicated, Hepa-filtered, humidified 37° C. 5% CO₂ incubator (step 409) for 7 days. At day 3 or 4, the bags are visually inspected to check for media color change or bacterial/fungal contamination (step 410). On day 7 (although the culture period may be as little as four days), the tissue culture bags may be removed from the incubator and samples removed therefrom for quality control, e.g., viability staining, microbial analyses, cell enumeration using a hematology analyzer (e.g., from Beckman-Coulter, Hialeah, Fla.), microscopic examination of dendritic cell morphology, and immunophenotyping to determine the purity of the dendritic cell preparation (step 411). Immunophenotyping may be performed using a flow cytometer (e.g., FACSCalibur, Becton Dickinson, San Jose, Calif.) and corresponding software (e.g., CellQuest, Becton Dickinson, San Jose, Calif.). The monoclonal antibody panel may include antibodies to CD45/CD14, CD3/CD19, CD1a, CD11c, HLA-DR, CD83, CD86 and CD123.

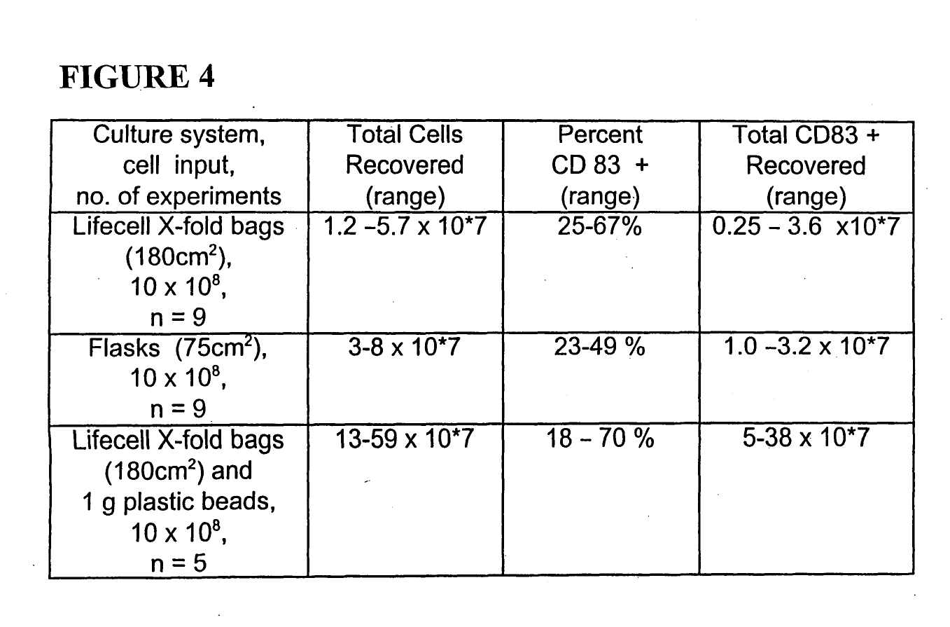

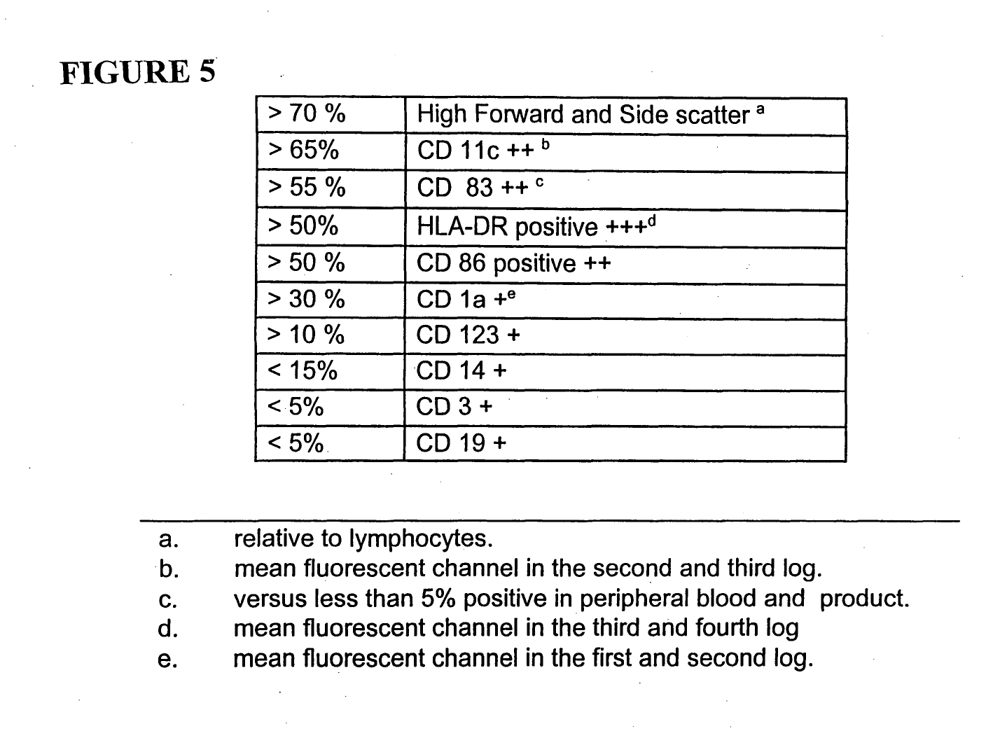

In experiments using the method described above, plastic beads were not visible in the supernatant on microscopic examination. The yields of DC are improved when compared to the other systems studied (see FIG. 4). The immunophenotype of the recovered cells (see FIG. 5) meets established DC phenotypes effective in adjuvant vaccine therapy. Culture supernatants are routinely negative for microbial contamination.

The quantities of the cells produced are acceptable for adaptive transfer strategies. Current tumor antigen vaccine protocols typically use approximately 10⁷ to 10⁸ total DCs. Using this closed system of culture, a sufficient number of DCs can be harvested for a complete course of therapy using a single 10-liter MNC apheresis and an average of five culture bags.

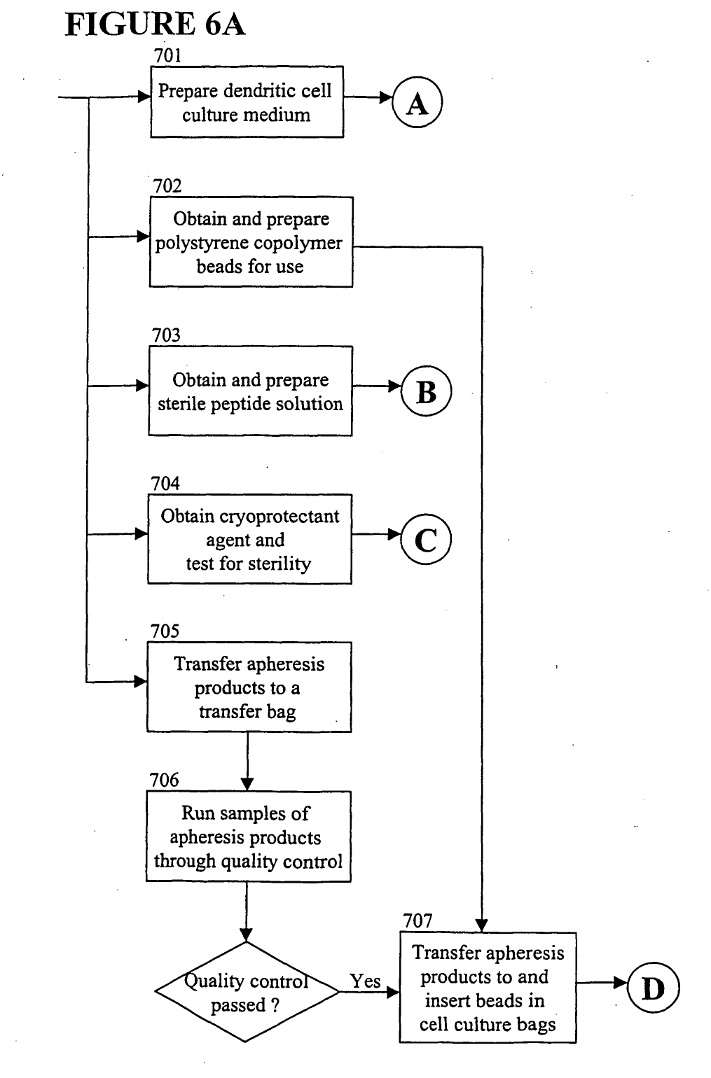

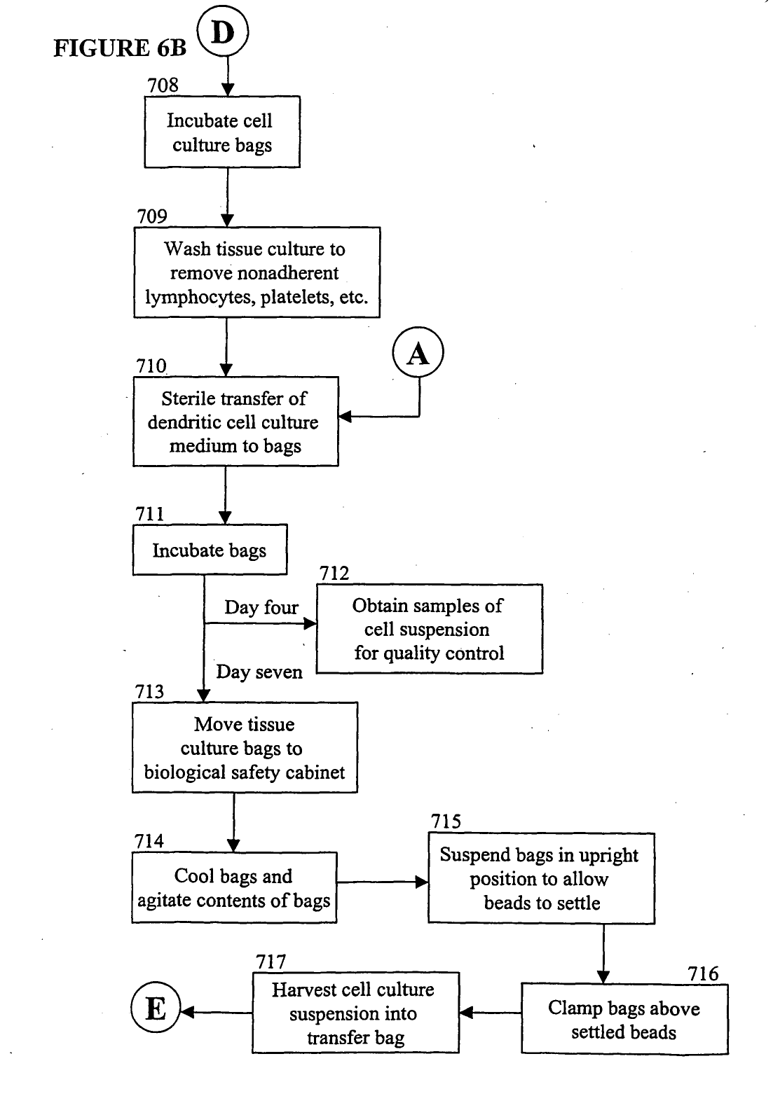

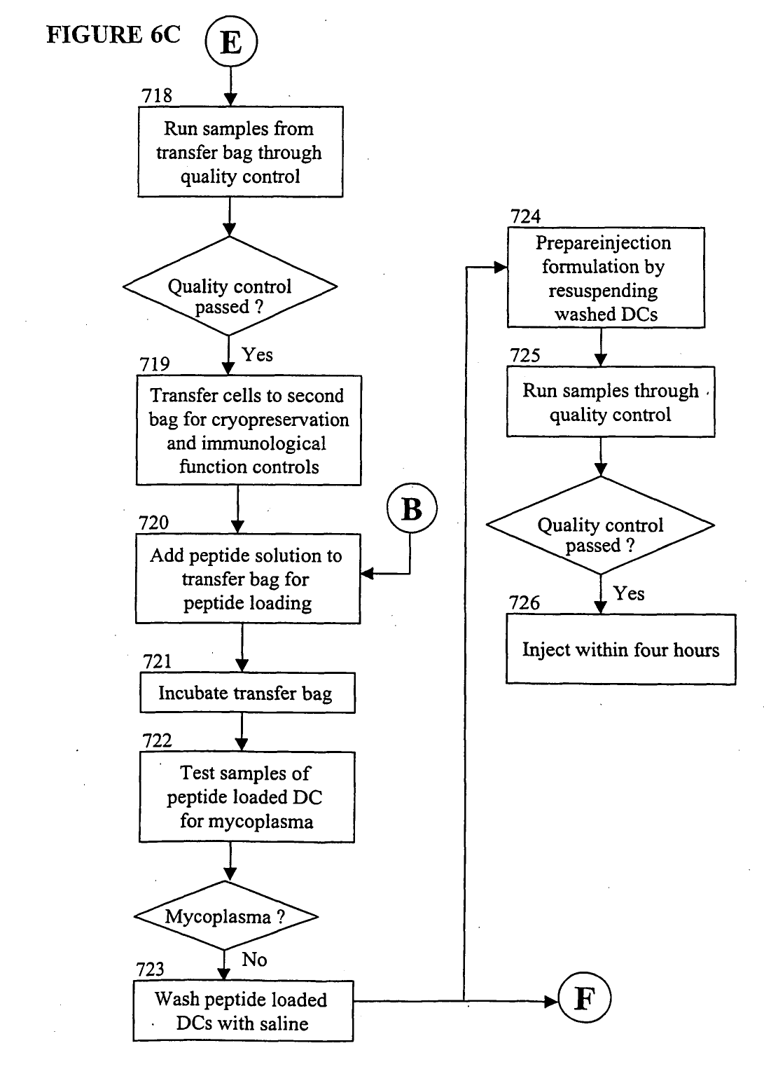

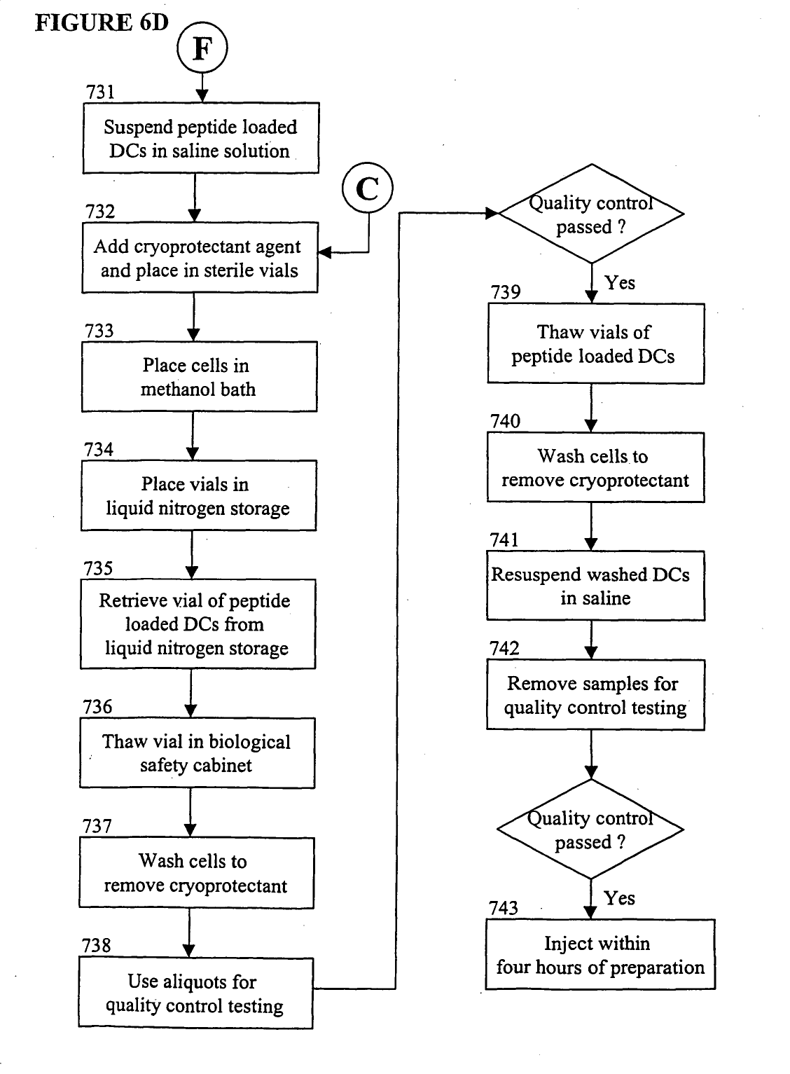

Another embodiment will be described with reference to FIGS. 6A through 6D.

A dendritic cell culture medium is prepared (step 701) by combining AIM V media (e.g., BB-MF 2557, Life Technologies, Grand Island, N.Y.), rh-IL-4 (e.g., 1000 U/ml, GLP grade, Sigma Aldrich, St. Louis, Mo.) and rh-GM-CSF (e.g., 25 ng/ml, Therapeutic grade, Immunex, Seattle, Wash.).

Polystyrene copolymer beads (e.g., 250 micron diameter, density=1.07 g/cm³, BB-MF 3094, Solohill Engineering, Inc., Ann Arbor, Mich.) are obtained and prepared (step 702) for use. For example, the beads may be suspended in a phosphate buffered saline (e.g., EDR9865, therapeutic grade, Nexell, Calif.) [100 gms beads/200 mls saline], placed in an autoclavable glass bottle and capped, and sterilized in an autoclave (e.g., 20 lbs/sq.in. at 121° C. for 1 hour with slow exhaust). The container is sealed and then transferred to a biological safety cabinet. A 1 ml aliquot is removed, placed in the transport tube, and tested for sterility (e.g., Bioscreen Testing Services, Inc., Torrance, Calif.). The polystyrene beads are used if no bacterial growth is detected.

Also, a sterile peptide (e.g., HER-2, or another peptide antigen specific to another target tumor) solution is prepared (step 703), using for example synthetic peptide (e.g., GLP grade, American Peptide Company) and phosphate buffered saline. For example, HER-2 synthetic peptide powder is dissolved in saline at a concentration of 200 μg/ml (20×) and sterile filtered through 0.2 micron nylon membrane. The solution is aliquoted in sterile 10 ml vials and stored.

A cryoprotectant agent (e.g., DMSO, USP grade, Gaylord Chemical Corporation, Slidell, La.) is obtained and tested for sterility (step 704).

Apheresis products are transferred to a transfer bag (step 705). Samples of the apheresis products are run through quality control (e.g., hematology analyzer, Trypan blue viability, CD45/14 immunophenotype)[step 706]. If quality control is passed, apheresis products (e.g., 10×10⁸ mononuclear cells/bag×four to five bags) are transferred (step 707) from the transfer bag using a sterile connecting device (e.g., Lifecell transfer set, Nexell Therapeutics, Irvine, Calif.) to gas permeable tissue culture bags (e.g., therapeutic grade, Lifecell X-fold Cell Culture Containers PL2417, 180 cm², Nexell). The beads that pass quality control (step 702) also are inserted in the bags.

The tissue culture bags then are incubated (step 708), for example, in a humidified 37° C., 5% CO₂ atmosphere for approximately four hours. At the midpoint, the bag is flipped from one side to the other. After four hours, the tissue culture is washed three times with AIM-V to remove nonadherent lymphocytes, platelets, grans, RBC, etc. (step 709). The wash includes transfer of the AIM-V media and expressing off the supernatant while leaving the beads in the bag.

Next, the dendritic cell culture medium (prepared in step 701) is transferred via a sterile process to the tissue culture bags (step 710). The bags are incubated again, for example, in a humidified dedicated 37° C. 5% CO₂ incubator (step 711) for 5 to 7 days. At day 4, samples of cell suspension are removed for quality control (step 712). On day 7, the tissue culture bags are moved from the incubator to a biological safety cabinet (step 713). The bags are cooled to room temperature, and the contents are gently agitated for five minutes (step 714). The bags are suspended in an upright position to allow the beads to settle for 5 minutes at 1×g (step 715), and the bag is clamped above the settled beads (step 716). The cell culture suspension is harvested (step 717) by expression into transfer bags (e.g., Stericell bags, Nexell Therapeutics) using a sterile connecting device (e.g, from Terumo Corp., Phoenix, Ariz.).

Samples are removed from the transfer bags and run through quality control (step 718). For example, if no beads are present and viability is greater than 95%, then the samples are passed to immunophenotype by flow cytometry. If quality control is passed, cells (e.g., approximately 50×10⁶ cells or any range, such as all of the cells) can be transferred (step 719) to a second bag for cryopreservation and immunological function controls (e.g., seven day proliferation assay using harvested cells as stimulators for lymphocytes from three different individuals).

HER-2 peptide solution is added to the transfer bag (step 720) for peptide loading onto the HLA Class I of the DC (final concentration is 10 ug/ml). The transfer bag is incubated (step 721) overnight (e.g., 4 to 12 hours at 37° C., 5% CO₂ in a humidified atmosphere of a dedicated incubator). Samples of the peptide loaded DC are tested for mycoplasma (step 722). If the test results are negative for mycoplasma, the peptide loaded DCs are washed three times with therapeutic grade phosphate buffer saline (step 723). In a preclinical phase, samples of the suspension may be removed for quality control analysis, such as for endotoxin (e.g., USP LAL), fluoride (ion specific electrode) and residual organic solvent (GC-MS).

Injection formulation is prepared by resuspending the washed DCs (step 724) at a concentration less than 10⁷ cells/ml (e.g., 3, 6, 9 or 12×10⁶ cells/ml) in saline supplemented with 5% autologous serum obtained the same day. Samples are removed for quality control (step 725), such as Gram stain. If quality control is passed, the injection formulation is cleared for administration and injected within four hours of preparation (step 726).

The remaining cells are cryopreserved. First, the peptide loaded dendritic cells (also DC without peptide) are suspended (step 731) in a solution of therapeutic grade saline supplemented with 5% autologous serum (5×10⁶/ml). Cryoprotectant agent is added to a final concentration of 10% and placed in sterile NUNC vials (5 ml) [step 732]. The cells are placed in a methanol bath at −70° C. overnight (step 733), then placed in vapor phase liquid N₂ storage until use (step 734).

After two to three days of storage, a vial of peptide loaded DC is retrieved (step 735) from the liquid N₂ storage for quality control testing. Vials are thawed at 37° C. in a biological safety cabinet (step 736). The cells are washed with AIM-V to remove cryoprotectant (step 737).

Aliquots are removed for the following assays:

| | |
|---|---|
| Viability | (>70%) |
| Sterility USP | (No growth) |
| Mycoplasma by PCR | (negative) |
| MLC test (7 day) | (Stimulate proliferative response |
| | greater that 3 × BACKGROUND at |
| | responder to stimulator ratio of |
| | 10 to 1) |
| Endotoxin (USP LAL) | <0.06 EU/ml |

If the quality control test (step 738) is passed, the cryopreserved DC are released for thawing. Vials of cryopreserved peptide loaded DC are thawed (step 739) at 37° C. in the biological safety cabinet. Cells are washed with therapeutic grade saline three times to remove cryoprotectant agent (step 740). Washed DC are resuspended (step 741) in saline supplemented with 5% autologous serum obtained the same day. Samples are removed for quality control testing (step 742), e.g., viability staining and Gram stain. If viability is greater than 70%, then the cells are passed to adjust cell concentration to less than 10⁷ viable cells/ml (e.g., 6, 9 or 12×10⁶ viable cells/ml)×1 ml. Next, a Gram stain is applied. If the Gram stain is passed, the injection formulation is cleared for administration and injected within four hours of preparation (step 743).

### Example 2

### Transcript Profiling of Human Dendritic Cells Maturation-Induced Under Defined Culture Conditions: Comparison of the Effects of Tumor Necrosis Factor Alpha, Soluble CD40 Ligand Trimer and Interferon Gamma

Using cDNA arrays, patterns of gene expression were characterized in populations of human dendritic cells (DCs) produced for clinical use. Culture and maturation induction of myeloid adherent cells under serum-free conditions yielded DCs with phenotypes similar to those described in serum-based systems. Analysis of gene expression in DCs treated with tumor necrosis factor alpha, soluble CD40L trimer or interferon gamma, however, showed specific patterns for each factor examined. Expression of several transcripts in DCs and/or differentially regulated according to the differentiation state of the DCs were documented, and suggest important functional differences among the DC populations examined. In addition, DC maturation directs changes in the levels of mRNA specific for transcriptional regulators that effect the production of cytokines (e.g., BCL-6, c-rel). Other changes observed, including alteration in the gene expression profile of adhesion molecules and chemokine receptors such as CD44H, CD 49B, Rants R, CXCR5 and CDS 7, suggest differences in trafficking potential between the populations studied. This broad-based description of DC populations, produced under serum-free conditions, provide better basis for definition of intermediate stages of DC maturation as well as the differentiation-inducing effects of cytokines on these cells.

Dendritic cells (DCs) are the most effective antigen-presenting cells (APCs) of the immune system characterized to date. These cells, following an encounter with an antigen, can stimulate both naive and memory T-cell responses (Banchereau et al., 2000). The current understanding of DC biology suggests, however, that the differentiation state of the dendritic cell qualitatively affects their interaction with T lymphocytes (Kalinski et al., 1999; Lanzavecchia, 1999). Depending on the level of maturation, DCs typically elaborate different profiles of chemokines and cytokines [e.g., interleukin 12 (IL-12)], show different antigen-processing abilities and have altered expression of adhesion receptors and co-stimulatory molecules (Thomas & Lipsky, 1994).

The use of dendritic cells as adjuvants in cancer immunotherapy is supported by studies showing that injection of tumor antigen-loaded dendritic cells can induce tumor-specific cytotoxic T lymphocyte (CTL) responses and, in some cases, regression of metastases. Clinical trials using dendritic cells as vaccination adjuvants have progressed to phase D efficacy studies (Dhodapkar et al., 1999, 2000; Brinckerhoff et al., 2000; Larsson et al., 2000; Rieser et al., 2000; Tjoa & Murphy, 2000). The populations of DCs used in these trials, however, have not yet been fully characterized.

Populations of human DCs can be produced for clinical use by culturing precursor cells in the presence of cytokines, notably granulocyte-macrophage colony-stimulating factor (GM-CSF) and IL-4 (Bernhard et al., 1995; Zhou & Tedder, 1996; Thurner et al., 1999). Populations of DCs produced with GM-CSF and IL-4 are phenotypically immature and characterized by active macropinocytosis, endocytosis (Sallusto et al., 1995) and submaximal expression of MHC gene family products and co-stimulatory molecules (Bender et al., 1996). Immature DCs can be driven to a mature phenotype following exposure to a variety of agents including tumor necrosis factor alpha (TNF α) (Siena et al., 1995; Romani et al., 1996), CD40L (Caux et al., 1994; Cella et al., 1996), monocyte-conditioned medium (Bender et al., 1996), heparan sulphate (Kodaira et al., 2000), bacterial and fungal polysaccharides, nucleic acids (Sparwasser et al., 1998; Granucci et al., 1999; Hartmann et al., 1999; Rescigno et al., 1999) and prostaglandin E (PGE) (Kalinski et al., 1997a).

This disclosure describes good manufacturing procedure (GMP)-compatible culture methods for the production of dendritic cells to be used in cancer immunotherapy (see also Maffei et al., 2000). The immunophenotype of DCs obtained by this method suggests that these DCs share characteristics with immature DCs previously described (Ye et al., 1996; Zhou & Tedder, 1996; Morse et al., 1997). The maturation state of DCs may affect how they interact with T cells (Kalinski et al., 1999; Tanaka et al., 2000; Vieira et al., 2000). It was sought to better characterize the DCs produced in this system and examine the phenotypes of DCs that were maturation-induced by recombinant TNF α, soluble CD40 ligand trimer (sCD40 LT), interferon gamma (IFN γ) and IL-7 (Takahashi et al., 1997; Li et al., 2000). Analysis of a broad array of transcripts from each of the DC populations studied revealed both similarities and differences in the patterns of gene expression. The data show that both immature and mature human DCs of a mixed DC₁-DC₂ phenotype can be produced under well-defined culture conditions. This study also suggests that, dependent on the culture conditions used, DC polarization can be partially selected, resulting in different patterns of DC chemokine, lymphokine and cell surface molecule transcript expression.

These differences may be exploitable for improved vaccination strategies in DC-based cancer immunotherapy.

Materials and Methods

A closed system for culturing populations of monocyte-enriched peripheral blood mono-nuclear cells (MNCs), using is flexible gas-permeable cell culture bags and sterile connecting devices, was used (Maffei et al., 2000). Apheresis MNC products were obtained with informed consent from healthy volunteers. All materials and reagents used for the apheresis and DC culture were sterile and/or endotoxin free (<0.5 Limulus amebocyte lysate U/ml) and approved by the Federal Drug Administration (FDA) for human use with the exception of the plastic beads, H-4 and Nycoprep media. The MNC products were harvested on a Spectra apheresis system (Spectra, COBE BCT, Lakewood, Colo., USA) using a cell collection program (Glaser et al., 1999). These products were additionally purified by buoyant density centrifugation over Nycoprep 1068 media (Boyum, 1983). The MNCs (10×8 total cells) were then diluted in 100 ml of AIM-V media (therapeutic grade, Gibco Life Technologies, Grand Island, N.Y., USA) and loaded into gas-permeable cell culture bags (Lifecell X-fold cell culture containers PL2417, 180 cm², Nexell Therapeutics, Irvine, Calif., USA) containing styrene co-polymer beads (1 g, 90-125 μm diameter, density=1·05 g/cm³, SoloHill Engineering, Ann Arbor, Mich., USA). The tissue culture bags were then incubated in a humidified 37° C., 5% CO₂ atmosphere. After 4 hours, the contents of the bag were gently resuspended, the beads allowed to settle and the supernatant was then removed. This procedure was repeated three times with 50 ml of AIM-V media (Gibco). After removal of non-adherent cells, 100 ml of AIM-V media containing recombinant human GM-CSF (rh-GM-CSF; 50 ng/ml, Sargramostim, Immunex, Seattle, Wash., USA) and rh IL-4 (1000 U/ml, Sigma, St. Louis, Mo., USA) or rh IL-7 (32 U/ml, Sigma) was introduced into the tissue culture bags (Romani et al., 1996). Neither fetal calf serum nor autologous human serum was added to the culture medium. The bags were placed into a dedicated, HEPA-filtered, humidified 37° C., 5% CO₂ incubator. On d 3 of culture, 50 ml of fresh AIM-V media with GM-CSF and IL-4 (or GM-CSF and IL-7 or GM-CSF alone) was added to each bag (cultures designated G4, G7 or G respectively). On d 6 of culture, fresh medium with cytokines was again added. In addition, the following maturation factors were added to DCs cultured in GM-CSF and IL-4: 25 ng/ml rh TNF α (R & D Systems, Minneapolis, Minn., USA) (cultures designated G4T), 5 μg/ml recombinant soluble CD40L trimeric fusion protein (a kind gift of Immunex Corporation) (cultures designated G4CD40L) or 1000 U/ml IFN γ-1b (Actimmune, Intermune Pharmaceuticals, Palo Alto, Calif., USA) (cultures designated G4IFN). On d 8, the cell suspensions were harvested, washed and pelleted. Aliquots of the cells were removed for phenotyping using immunofluorescent flow cytometry and the remaining cells were used for RNA isolation. Approximately 10 ml of cell culture was retained in the bags and additional fresh medium containing only GM-CSF (25 ng/ml) was added. A repeat immunophenotyping was performed on d 10 of culture. Cell pellets were stored at −80° C. until use.

Total RNA was prepared from cell cultures initiated with apheresis products from three different individuals using the TREZOL Reagent (GibcoBRL) according to the manufacturers recommendations. To completely remove RNases, the RNA samples were phenol-chloroform extracted twice. Samples were then precipitated and resuspended in RNase-free water. To eliminate potential genomic DNA contamination, an aliquot of 50 μg of RNA for each sample was incubated with 5 units of RQ1 RNase-free DNase (Promega, Madison, Wis., USA) for 30 min at 37° C., using the buffer recommended by the manufacturers. Finally, the RNA samples were again phenol-chloroform extracted, then precipitated and resuspended in RNase-free water at a concentration of 1 μg/μl. The quality of the RNA prepared was confirmed by analyzing the samples by electrophoresis on a 1·6% agarose gel in Tris-Acetate-EDTA buffer. RNA samples were stored at −80° C. until further use.

Large-scale expression probing of immature and maturation-induced human dendritic cells was performed using the Atlas Human Hematology/Immunology cDNA Expression Arrays (Clontech Laboratories, Palo Alto, Calif., USA). Each nylon membrane array was spotted in duplicate with cDNA fragments representing 408 known genes and several housekeeping genes or control sequences. Each cDNA fragment was 200-600 bp long and was amplified from a region of the transcript that lacked the poly A tail, repetitive elements or other highly homologous sequences, to minimize cross-hybridization and the non-specific bindings of the cDNA probe. A list of these genes, including array coordinates and GenBank accession numbers, is available (Clontech Laboratories, 2001). For side-by-side array hybridizations, 5 μg of total RNA from each cell population was reverse-transcribed in the presence of 5 μl of [γ-³²P]-dATP (111 TBq/mmol; 370 kBq/ml) (Amersham Pharmacia Biotech; Arlington Heights, Ill., USA) using the reagents and the protocol provided in the Atlas cDNA Expression Array kit to synthesize [³²P]-radio-labeled cDNA probes. Radiolabeled probes were denatured under basic conditions, neutralized in the presence of 5 μg of Cot-1 DNA (GibcoBRL) and then added to separate 5 ml aliquots of ExpressHyb hybridization solution (Clontech) containing 100 ng/ml of heat-denatured sheared salmon testes DNA (Sigma), to reach a final probe concentration of approximately 2·5×10⁵ cpm/ml. Hybridization/cDNA probe solutions were applied to prehybridized Atlas Array membranes (1 hour in ExpressHyb with 100 ng/ml of heat-denatured sheared salmon testes added at 68° C. in the absence of a labeled probe) and hybridized overnight at 68° C. After hybridization, membranes were washed twice with 200 ml of 2× saline sodium citrate (SSC), 1% sodium dodecyl sulphate (SDS) solution at 68 T for 30 min. followed by two 30-min washes in 200 ml of 0·1× SSC, 0·5% SDS, at 68° C. Finally, the membranes were rinsed in 2×SSC and exposed overnight to phosphor screens.

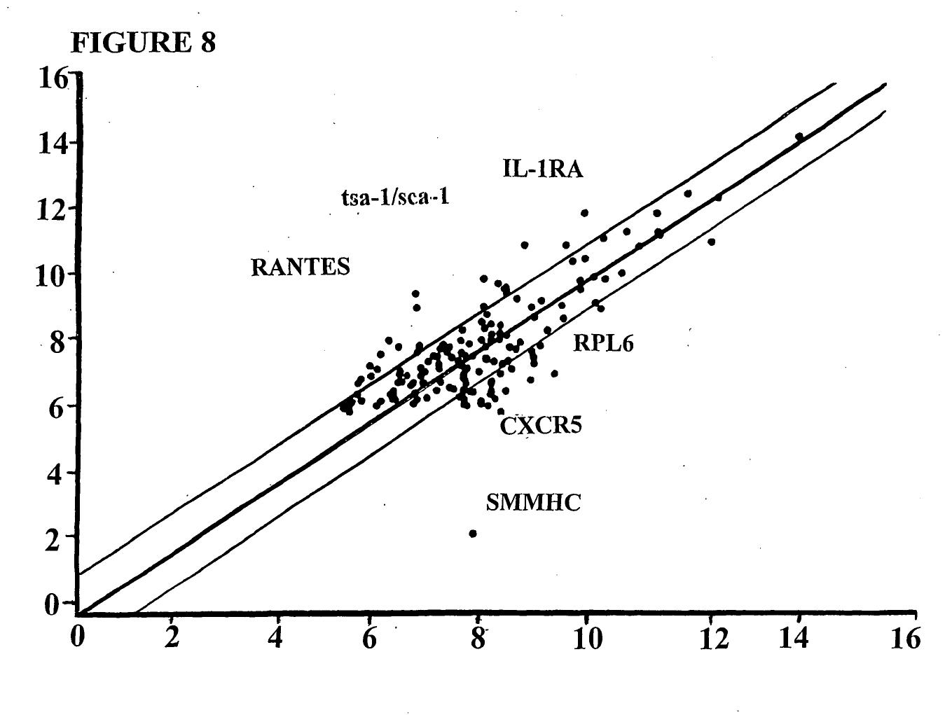

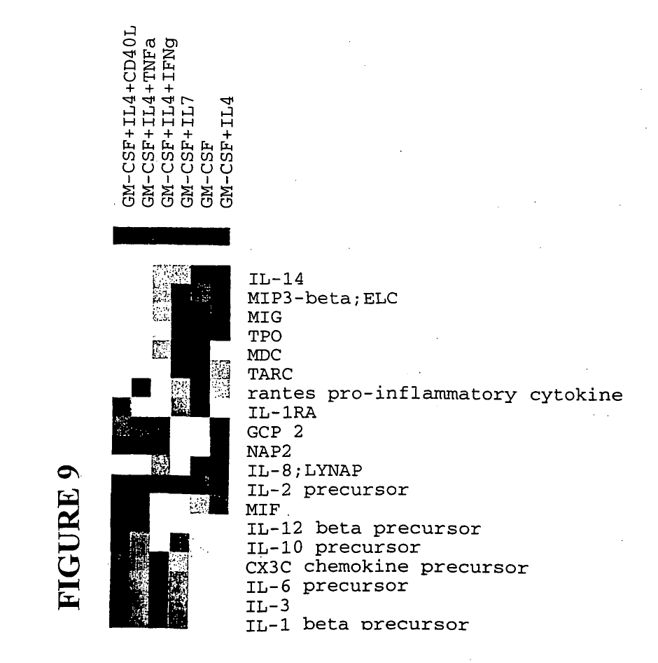

Hybridized Atlas Arrays were visualized and quantified using a PhosphoImager (Molecular Dynamics, Sunnyvale, Calif., USA) at a pixel resolution size of 80 μm. A grid matrix was generated and applied to the phosphoimage of each Atlas Array, which identified the duplicated target location for each of the 406 known genes as well as the nine genes defined as housekeeping genes and the 12 negative control sequences. The intensity of hybridization signal for each gene sequence was the average of the values determined for both spots in the target location and corrected for background using the intensity values of pixels surrounding the spot areas. Calculated intensities correlated linearly with the amount of message in the total RNA sample, as the target cDNA fixed to the membrane was in excess and the backgrounds were sufficiently low. For assessing differences in gene expression between arrays, the intensity values of each known gene were normalized to the intensity of designated housekeeping genes [i.e. glyceraldehyde phosphate dehydrogenase (GAPDH) and hypoxanthine phosphoribosyl transferase (HPRT)]. These two genes were selected for normalization over other so-called housekeeping genes (e.g., HLA-C and cytoplasmic β actin), which had been demonstrated to change expression levels during differentiation of DCs in preliminary experiments. Following this normalization, the sums of the intensity values of all the genes on a given array were within one standard deviation of the mean summed intensity for all arrays studied. Comparison between mRNA populations of various DC populations was performed on Atlas arrays of the same batch. Genes that showed an average increase or reduction of greater or equal to fourfold were tabulated. As a measure of consistency in gene expression analysis a scatter plot analysis, in which each point represents a particular gene and its coordinates, as determined from its normalized expression value, was performed (FIG. 8). In each scatter plot, points that lie close to the main diagonal represent genes that are expressed at similar levels in the various cell populations studied. For genes that lie away from the diagonal, the perpendicular distance from the diagonal represents the degree of differential expression between the two populations studied. The data were analyzed and displayed (Eisen et al., 1998). Briefly, the hierarchical clustering methodology produces a table of results wherein the elements of the array representing specific genes are grouped based on similarities in their patterns of gene expression (FIGS. 9-13). The same methodology was then applied to cluster the data from each population of dendritic cells according to the similarities in their overall patterns of gene expression. The data tables are presented graphically as black and white images. Along the vertical axis, the genes analyzed are arranged as ordered by the clustering methodology, so that the genes with the most similar patterns of expression are placed adjacent to each other. Along the horizontal axis, experimental samples are similarly arranged such that those with the most similar patterns of expression across all genes are placed adjacent to each other. The grey scale value of each square in the table image represents the measured expression of each gene. Where grey scales values are presented, white represents a high level of expression relative to lower levels (indicated in dark purple).

After DNase I treatment, 1 μg of total RNA from each sample was used as template for the reverse transcription reaction. cDNA was synthesized using Oligo(dT)₁₅ primer and AMV reverse transcriptase (Reverse Transcription System from Promega, Madison Wis., USA). All oligonucleotides primers for the semi-quantitative polymerase chain reaction (PCR) were synthesized by Life Technologies, Rockville, Md., USA. The following primers were used:

| | | | |
|---|---|---|---|
| (a) | GAPDH, | AACGGATTTGGTCGTATTGGGC | (G3-F) |
| and | | | |
| | | TCGCTCCTGGAAGATGGTGATC | (G3-R); |
| (b) | BCL-6, | CCTTAATCGTCTCCGGAGTCG | (BCL6-F) |
| and | | | |
| | | CCATCTGCAGGTACATAGCCGT | (BCL6-R); |
| (c) | CD37, | TTTGTGGGCTTGGCCTTCGTGC | (CD37-F) |
| and | | | |
| | | TAGGATTGTGGAGTCGTTGGTCGCC | (CD37-R); |
| (d) | CD81, | GCGCCCAACACCTTCTATGTAGGC | (CD81-F) |
| and | | | |
| | | AGCACCATGCTCAGGATCATCTCG | (CD81-R); |
| and | | | |
| (e) | CD53, | GCTGGGCAATGTGTTTGTCATCG | (CD53-F) |
| and | | | |
| | | CAATCTGGCAGTTCAGGGTCAGTGC | (CDS3 F). |

The PCR reaction was performed in 30 μl with 20 mmol/l Tris-HCl (pH 8·4), 50 mmol/l KCl, 1·5 mmol/l MgCl2, 200 μmol/l of each dNTP, 20 units of recombinant Taq DNA polymerase/ml (PCR SuperMix from Life Technologies), in the presence of 100 pmol of each of the two appropriate primers. The same conditions were used for GAPDH, CD37, CD53 and CD81: the reaction mix was denaturated for 5 min at 94° C., followed by a program consisting of three steps, 40 s at 94° C., 40 s at 55° C. and 90 s at 72° C. These conditions were used for either 20 or 30 amplification cycles (Ferrer et al., 1998).

The PCR with the primers for BCL-6 was performed under similar conditions, substituting an annealing temperature of 64° C. rather than 55° C. To ensure the correct conditions for the semi-quantitative PCR, it was necessary to determine the optimum amount of cDNA and number of cycles for linear amplification. To this end, reverse transcription polymerase chain reaction (RT-PCR) was performed with the GAPDH primers on all samples, testing three different amounts of starting total RNA (1, 2·5 and 5 μg) and analyzing, for each amount, the PCR products obtained with three different number of cycles (10, 20 and 30) (data not shown). Linearity was preserved when 1 μg of total RNA was used as starting material for cDNA synthesis and 1/20th of the reaction product was used for PCR. Electrophoresis of the PCR product was performed on a 2% agarose gel containing 1 μg/ml of ethidium bromide. Images from the ethidium bromide-stained gel were captured with a Kodak DC120 Zoom digital camera and light intensity of the bands was quantified using Kodak Digital Science ID image analysis software (Eastman Kodak, Rochester, N.Y., USA).

Immunophenotyping of the cultured cells was performed using a FACSCalibur flow cytometer and CellQuest software. The monoclonal antibody (mAb) panel used included fluorochrome-conjugated antibodies to CD45/CD14, CD3/CD19, CD1a, CD11c, HLA-DR, CD83, CD86 and CD123. Staining, washing and analysis was performed as per manufacturers recommendations (Becton Dickinson).

Results

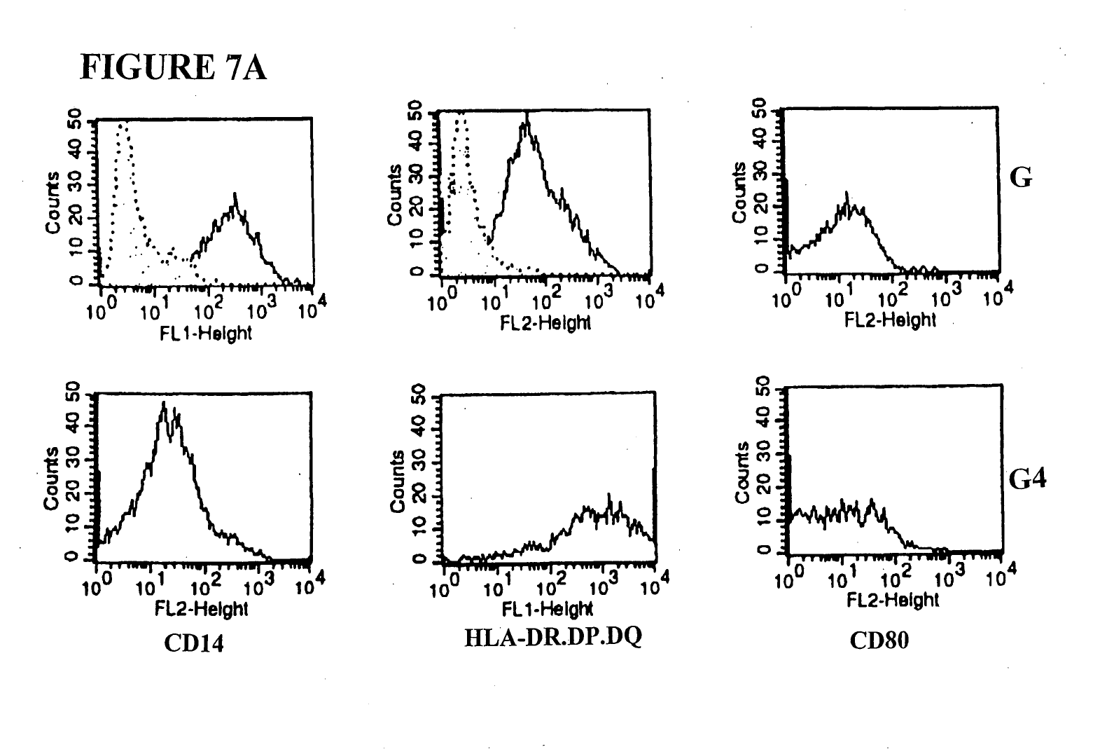

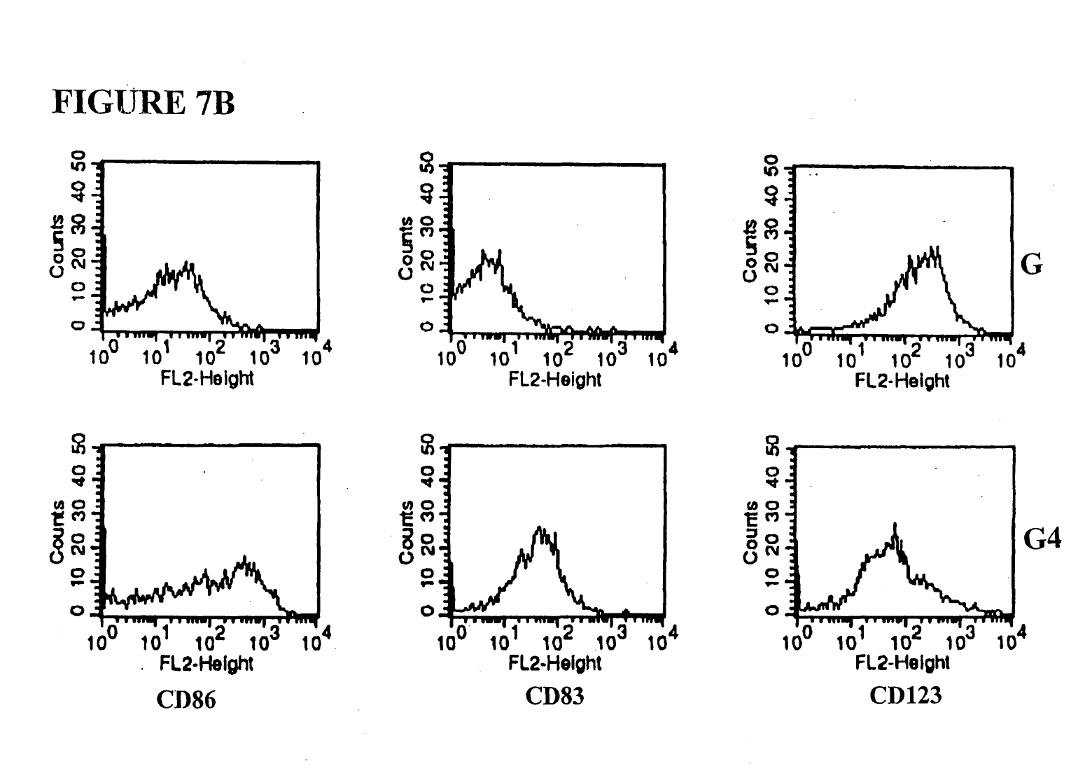

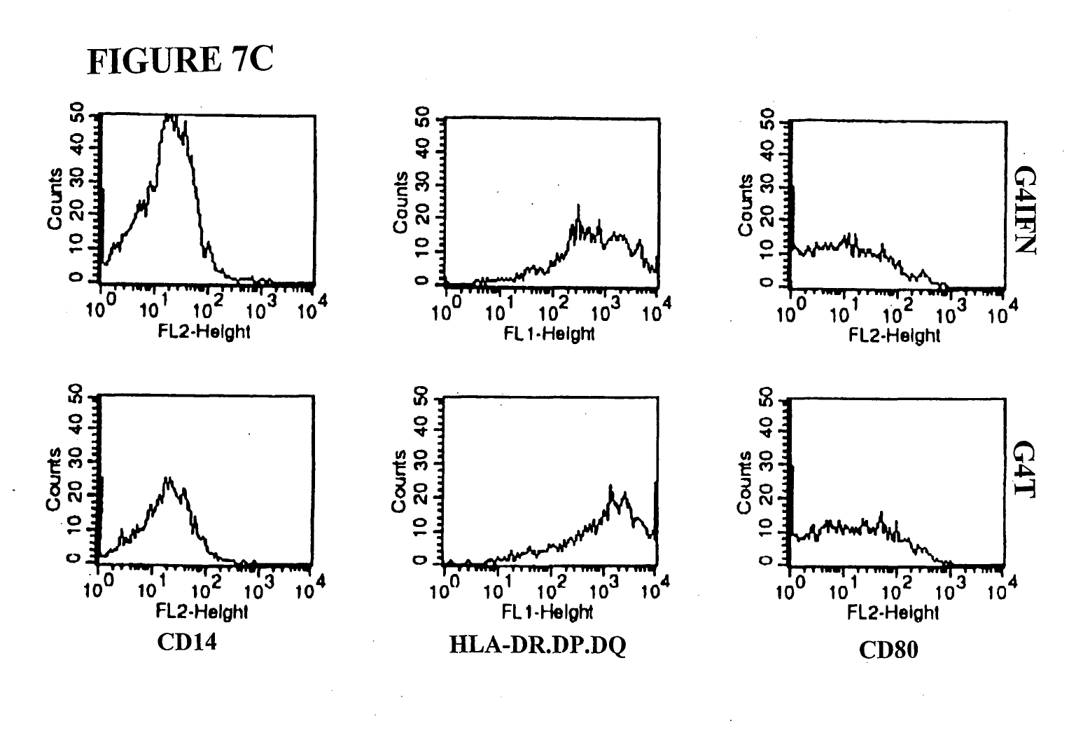

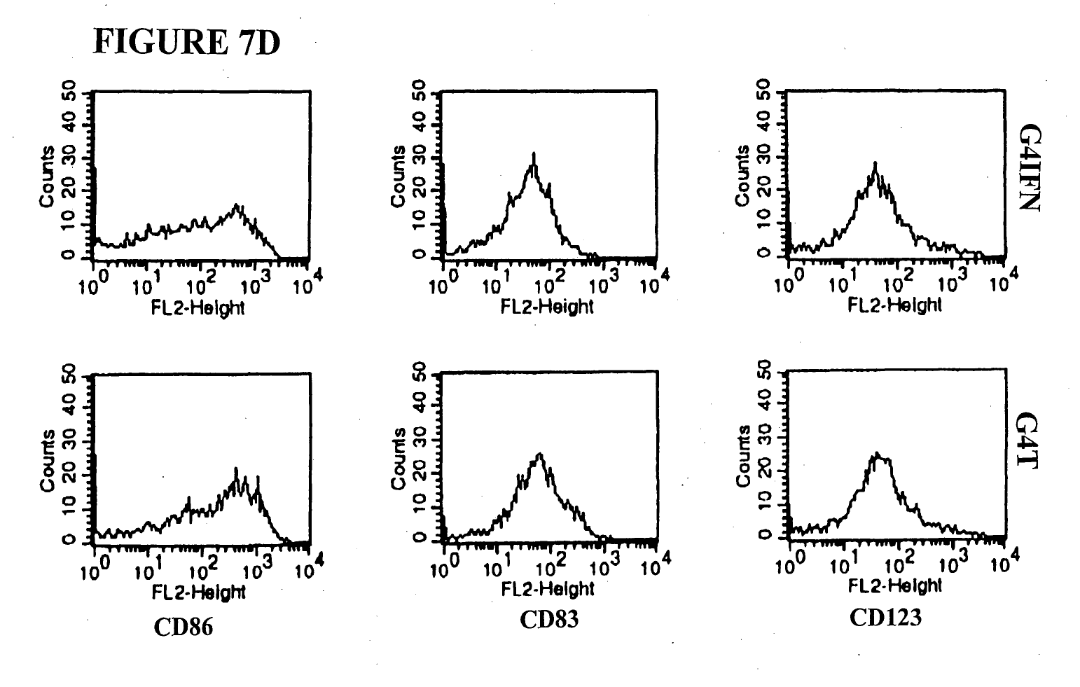

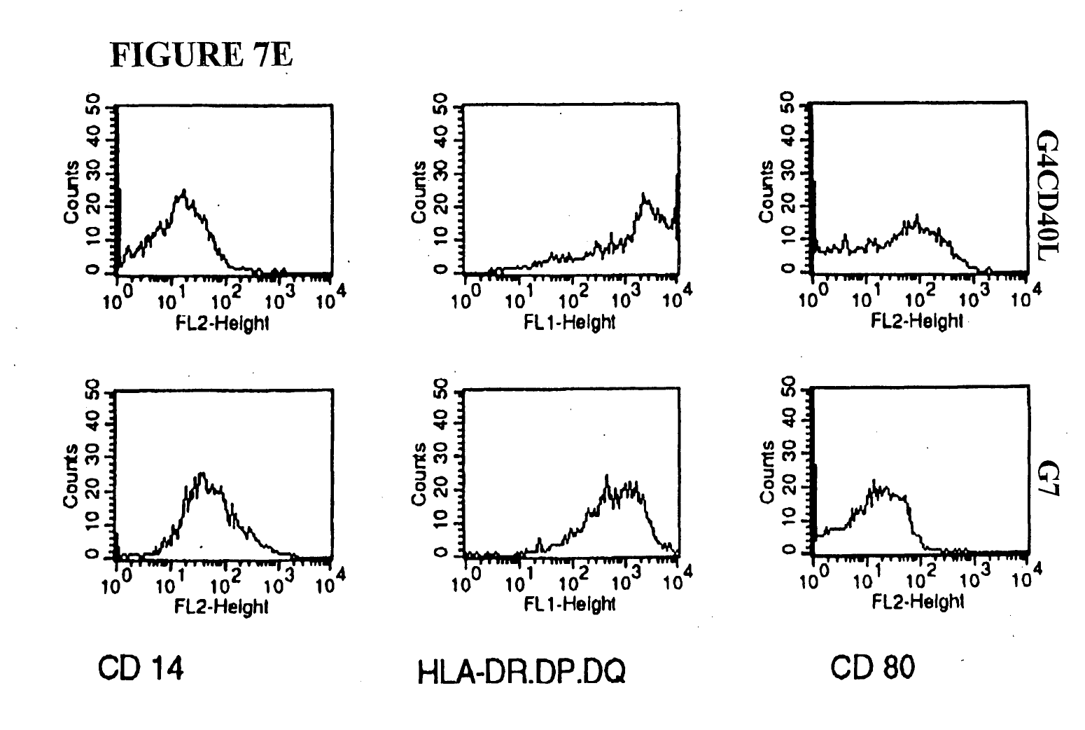

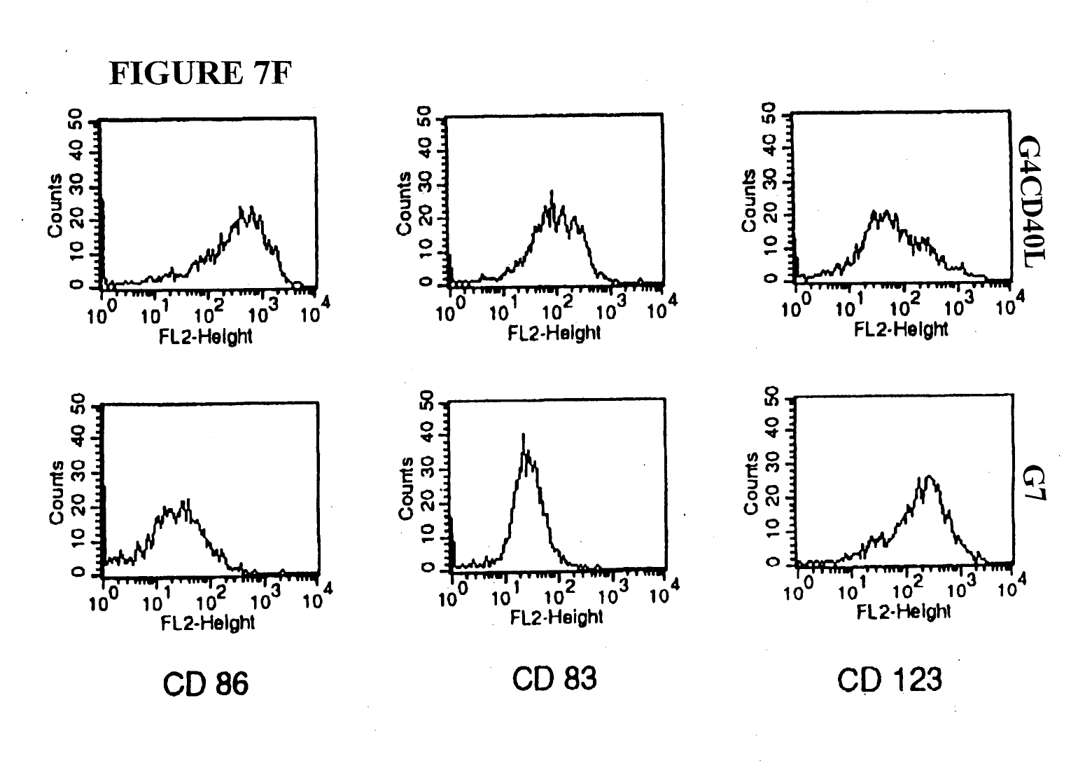

The dendritic cell populations obtained from the culture bags were immunophenotyped using a series of fluoro-chrome-conjugated mAbs. The data are shown as histograms in FIGS. 7A-7F depicting the number of cells exhibiting various fluorescence intensities. The dotted lines represent isotype-matched negative control antibody. The solid lines represent staining with specific antibodies. Results are representative of two independent experiments.

Flow cytometric measurements showed nearly unimodal distributions of the cell surface markers studied. Monocytes cultured in GM-CSF were CD 14 high, HLA class II low, CD 80 negative, CD 86 low, CD 83 negative and CD 123 positive (Row G). The same cell populations cultured for 8 d with GM-CSF and IL-4 (Row G4) or GM-CSF and IL-4 plus IFN γ (Row G4IFN) lost expression of CD 14 and CD 123, but displayed enhanced expression of HLA class II, CD 86 and CD 83 (Rows G4 and G4IFN). This is consistent with the immunophenotype of immature dendritic cells previously described (Romani et al., 1996). Cells cultured with GM-CSF and IL-7 (Row G7) displayed an immature immunophenotype with intermediate expression of CD 14, CD 83, CD 123, but a high level of MHC class II, and a low level of CD 80 or CD 86. Immature dendritic cells, following culture in either TNF α (Row G4T) or s CD 40L trimer (Row G4CD40L), showed further enhanced expression of HLA class II, CD 80, CD 86 and CD 83. The cell populations studied contained no CD 3-, CD 19-, CD 20- or CD 56-positive cells (data not shown). The immunophenotype of IFN γ, s CD 40L trimer and TNF α-treated cells was repeated on d 10. On d 10 these mature DCs maintained a similar pattern of expression of the cell surface markers studied (data not shown).

The arrays used in these experiments displayed 406 genes, of which 40% were expressed in the dendritic cell populations studied. In a scatter plot analysis in which different mRNA preparations from different dendritic cell populations were compared, the profiles and levels of the expressed genes represented in each population were similar (FIG. 8).

Many of the specific transcripts measured in DCs were distributed along the diagonal line of ‘identity.’ This indicates that cell culture, RNA isolation, reverse transcription for probe preparations and hybridization conditions were reproducible. To identify genes that were differentially expressed in the DC populations studied, hybridizations of cDNA probes synthesized from RNA isolated from all populations of maturation-induced DCs and immature DCs were compared side-by-side.

A scatter plot comparison of the gene expression data from monocytes cultured in GM-CSF (G) and immature DCs obtained from cultures containing GM-CSF and IL-7. (G7) is shown in FIG. 8. A pair-wise comparison of gene expression was performed by XY-scatterplot analysis of log base 2-transformed expression data. Expression profiles were obtained from monocytes cultured in GM-CSF alone and monocytes cultured with GM-CSF and IL-7. Each point represents the normalized expression of an individual gene within both mRNA populations. The thick line represents a predicted line of identity. The thin lines indicate thresholds of greater than twofold or less than one-half expression ratios.

Although many of the expressed genes lie relatively close to the diagonal line of ‘identity’, other genes exhibited a greater than twofold change in expression levels (FIGS. 15-19). The marked differences in gene expression profiles between immature and mature dendritic cells were corroborated by the pattern of expression of many genes whose regulation in DCs and monocytes have been previously characterized (Hashimoto et al., 1999, 2000). For the analysis performed in this study, these genes can be considered as control ‘sentinel genes’ (e.g., TARC, MDC, SMMHC, etc.). In addition, no T- or B-cell lineage-specific transcripts were detected in these arrays (e.g., CD 3, CD 152, CD 7, CD 19, CD 20).

The data presented in FIGS. 15-19, summarizing the most differentially expressed genes, are a subset of the larger data set that includes genes that showed smaller but significant changes in specific mRNA levels. To better understand the relationships between the different growth conditions (i.e. GM-CSF cultured monocytes, GM-CSF and IL-4, GM-CSF and IL-7, GM-CSF and IL-4 plus IFN γ, GM-CSF and IL-4 plus TNF α GM-CSF and IL-4 plus s CD40 LT), gene expression and phenotype of the dendritic cells produced, the cDNA hybridization data were analyzed using hierarchical cluster analysis.

Nineteen genes known to act as soluble immune mediators were selected from the larger group of expressed genes and analyzed by clustering (FIG. 9). Data were measured by using a cDNA array with elements representing approximately 400 distinct human genes. Genes were selected for this analysis if their expression level deviated from background by at least threefold in one or more of the different conditions studied. The dendrograms and tables were calculated as described in the text: the grey scale ranges from dark purple (lowest levels of expression) to white (highest levels of expression) with grey values indicating intermediate levels of expression. Each gene is represented by a single row of boxes a single column represents each culture condition. Clustering (and correlation statistics) was performed on groups of genes with known functions of chemokines and cytokine (r=0.95).

Examination of the dendrogram (x axis in FIG. 9) obtained by cluster analysis shows that the cell populations studied could be categorized into two main families. The first family, GM-CSF cultured monocytes (G) and immature DCs (G4 or G7) were characterized by their increased expression of IL-12 beta, CX3C chemokine, IL-6, IL-3 and IL-1 beta. Cluster analysis shows a significant increases in expression of MDC, TARC, Rants, IL-1 RA and IL-10 genes in G4 DCs relative to the GM-CSF cultured monocytes (FIGS. 9 and 15).

Similar to the intermediate phenotype revealed by FACs analysis, the pattern of gene expression in DCs grown in GM-CSF and IL-7 occupied an intermediate position in the dendrogram. These cells showed increased expression of MIF, IL-8, NAP-2 and GCP 2 relative to DCs obtained from all other culture conditions.

A second cluster observed in mature DCs (treated with either TNF α or s CD40 LT), showed increased expression of IL-14, MIPS beta, MIG, TPO, MDC and TARC. The expression of these genes were further increased beyond the levels seen in immature DCs and, relative to the other populations studied, reached the highest levels (designated in white in FIG. 9). Increased quantities of transcripts for TARC, Rants, IL-1 receptor antagonist, IL-10 and MIF were detected in DCs grown in GM-CSF, IL-4 and induced with IFN γ.

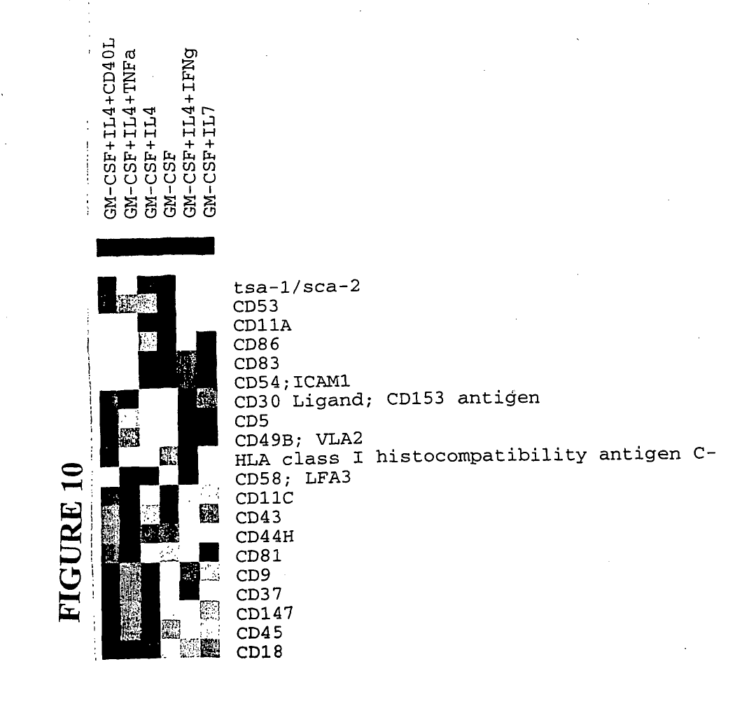

Twenty genes with known functions in cell-to-cell contact and/or that are involved in APC-effector cell communication were selected from the larger group of expressed genes and analyzed by clustering (FIG. 10). The gene expression patterns in the various cell populations studied could be categorized into three families. The first family, GM-CSF cultured monocytes (G) and immature DC (G4) were characterized by their increased expression of CD30L, CD 5 and CD 49B. Cluster analysis did not show a significant elevation of CD 83 and CD 86 gene expression in immature DCs (G4) relative to the cultured monocytes (G) (FIG. 15). A second family, formed by G4IFN and G7 DCs, were characterized by increased expression of tsa-1/sca-1, CD 53, CD 11a, CD11c, CD 44H and CD 147. DCs with the highest levels of MHC class II protein expression (FIG. 7) (G4T and G4CD40L) were clustered together on the basis of their increased expression of CD 11a, CD 86, CD 83 and CD54/ICAM1 (FIG. 10, r=0.91).

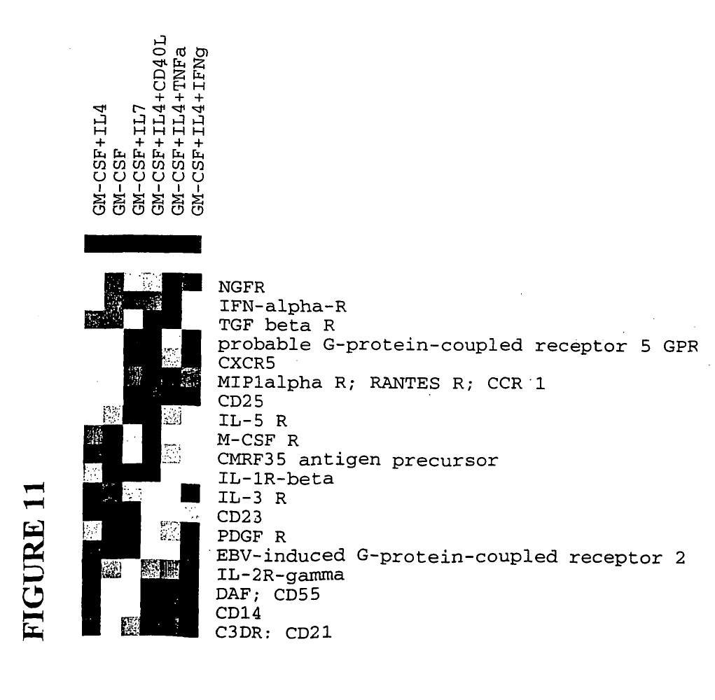

The expression of 19 molecules representing various receptors for cytokines, chemokines and lymphokines were also examined (FIG. 11). GM-CSF cultured monocytes and immature DCs (G4) could be distinguished on the basis of their increased expression of GPR5, CXCR5, MIP1α receptor, CD25 and IL-5 R. GM-CSF cultured monocytes and those cultured with additional IL-7 were grouped on the basis of increased expression of IL-2 R gamma subunits, CD 14 and CD 55/DAF, CD 21 and loss of BLR1/CXCR5 expression (FIGS. 11 and 16, r=0.92).

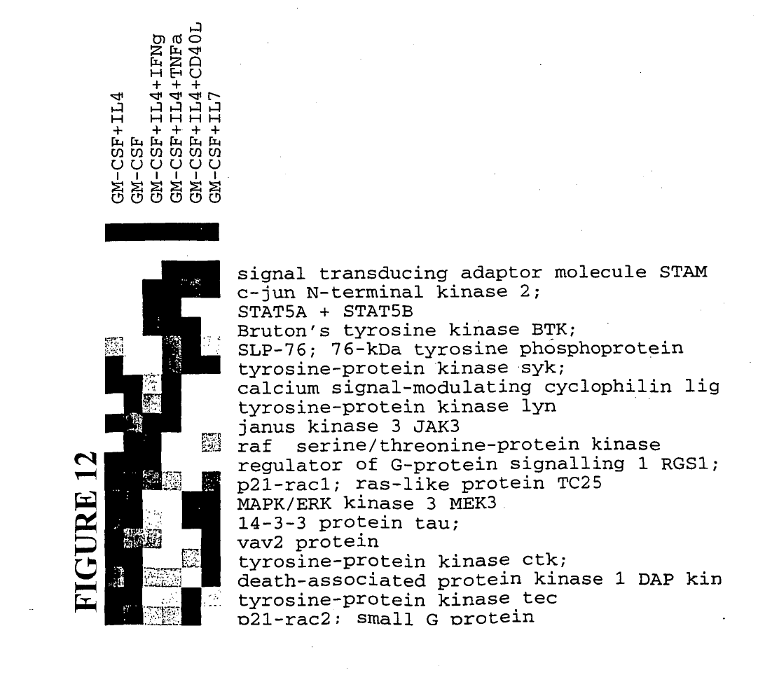

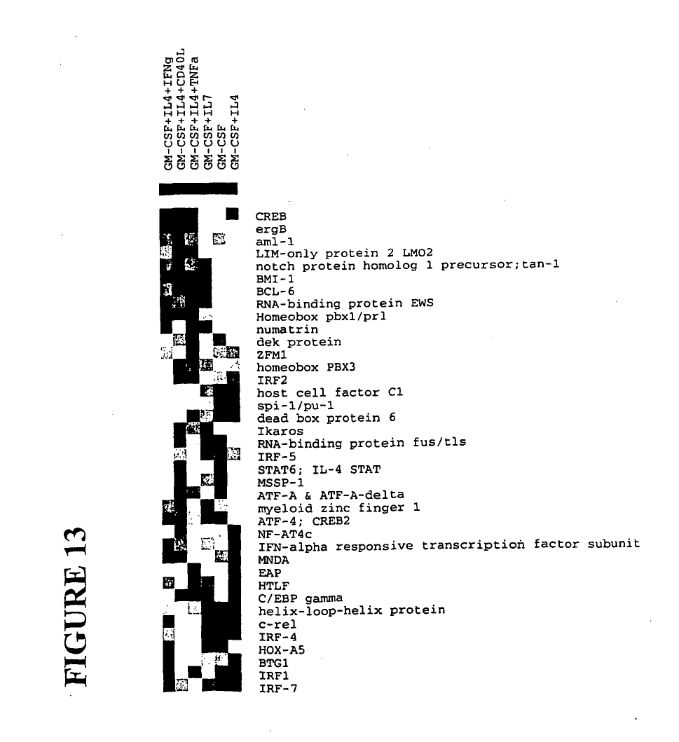

The expression of 19 molecules representing various kinases and G proteins (FIG. 12, r=0.97) and 39 transcription factors (FIG. 13) were analyzed using the above methods. While the relationship between receptor signaling and post-translational modifications of various kinases is well known, the relationship between receptor signaling and the expression of many signaling intermediates remains unexplored in DCs. GM-CSF cultured monocytes and immature dendritic cells (G and G4) clustered together by virtue of their common increased expression of STAM, jnk 2, STAT 5a+STAT 5b, BTK and SLP-76. Mature dendritic cells (G4T, G4IFN and G4CD40L) and those cultured in IL-7 were grouped together. DCs induced with INF γ or TNF α showed increased expression of MEK3, 14-3-3 tau, vav 2, ctk, DAPK1, tec, p21-rac2. An additional cluster formed by DCs treated with s CD40LT and G7 showed increased expression of CAML, lyn, JAK3, raf and RGS1.

Although the gene expression patterns of the transcription factors were complex, correlation among gene expression and DCs maturation could be discerned (FIG. 13, r=0.99). Monocytes cultured in GM-CSF and immature DCs (G7) formed one cluster, displaying increased expression of erg B, aml-1 and LM02. In cultured monocytes (G) and G4 DCs, tan-1, BMI-1, BCL-6, EWS, homeobox protein prl and numatrin showed higher expression.

The pattern of transcription factor expression in maturation-induced DCs (G4T, G4CD40L and G4IFN) appeared to be more specific for the type of DC, although the hierarchical cluster analysis did group DCs treated with sCD40L T and TNF α together. In this grouping, these mature DCs showed enhanced expression of c-rel, homeobox protein HOX-A5, helix loop helix protein, IRF-4 and spi-1/pu-1. Notably, this group also showed decreased expression of transcriptional regulators such as BCL6, AML-1 and CREB. The pattern of down-regulated BCL6 expression in human DC maturation-induced with s CD40 LT was confirmed in parallel experiments using the cDNA array methods previously described (Eisen et al., 1998), in which the patterns of gene expression in G4 DCs and DCs maturation induced with s CD 40 LT were compared (data not shown).

DCs, maturation-induced by INF γ, showed a unique cluster of increased gene expression formed by homeobox pbx3, IRF2, host cell factor cl, spi-1/pu-1, dead box protein 6, Ikaros and IRF 5, and C/EBP gamma.

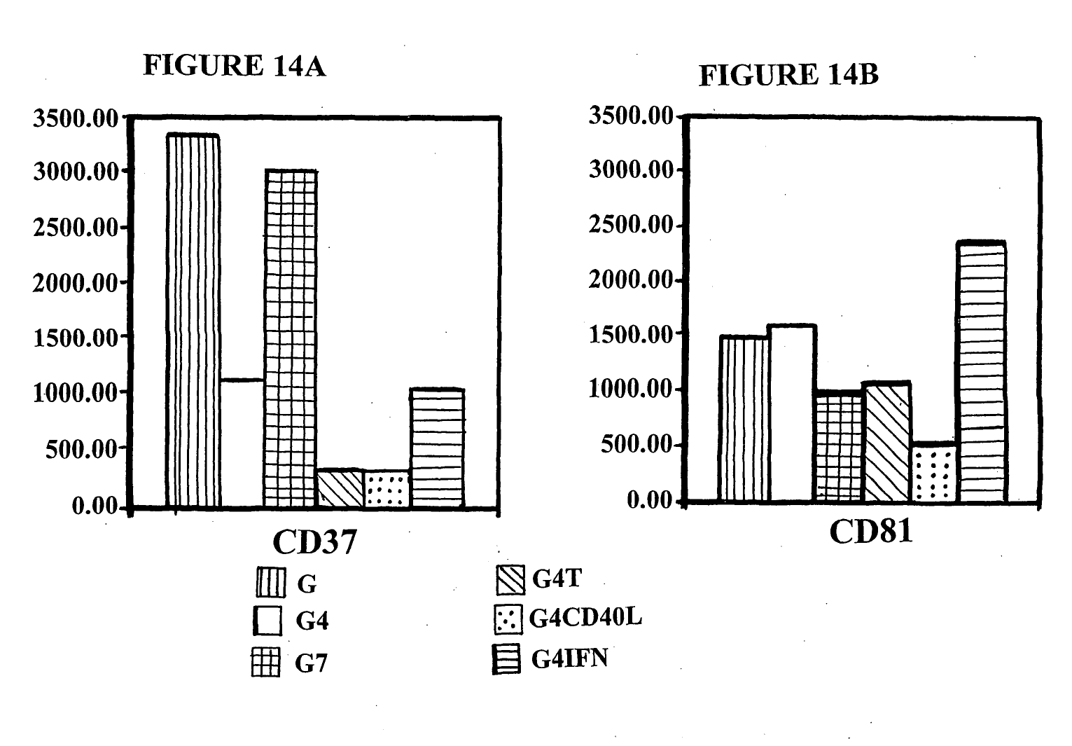

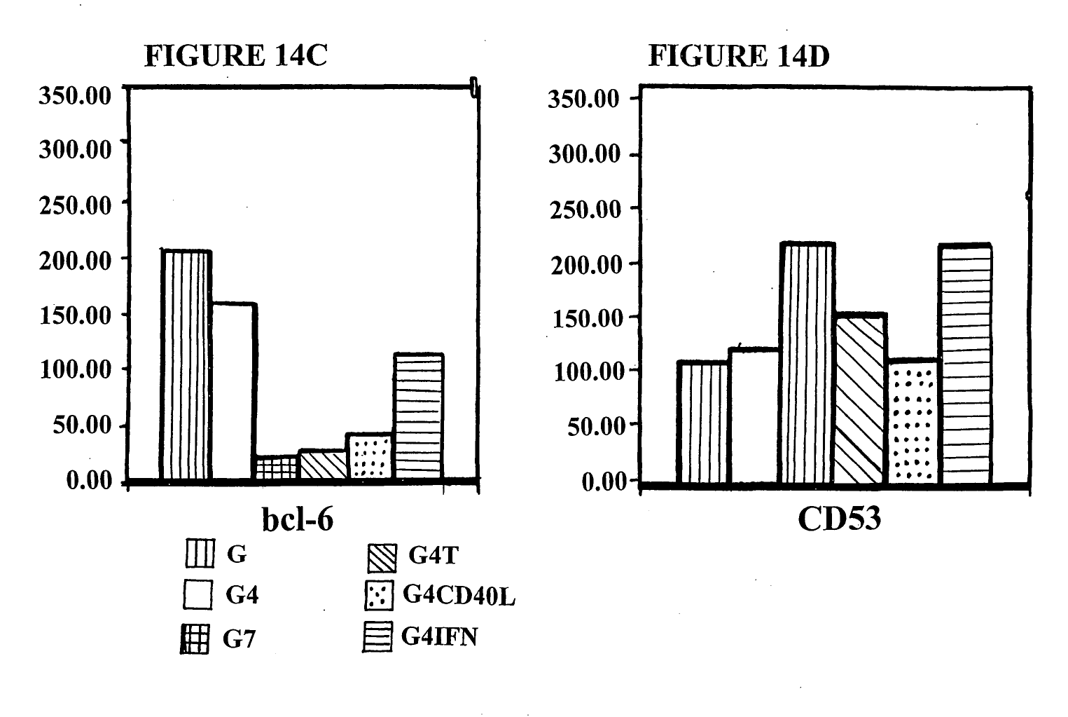

To confirm the results on the identification of differentially expressed genes obtained by cDNA array hybridization, the level of expression was determined using semi-quantitative RT-PCR of four genes (CD37, CD81, CD53 and BCL-6) whose expression in mature and immature DCs had not been previously characterized (FIGS. 14A and 14B). All band intensities were normalized to the expression of GAPDH, the same internal control used for the normalization in the array experiments.

When the cDNAs from immature and maturation-induced DCs were tested with the CD37 primers, an abundant accumulation of specific transcripts in cultured monocytes (G) and immature G7 DCs (G7), as well as a net decrease in the amount of this mRNA in the mature DCs treated with CD40L (G4CD40L) and TNF a (G4T) was detected. These results parallel the cDNA array hybridization findings. The PCR amplification with the CD 81 and CD 53 primers also confirmed the results obtained with the cDNA hybridization on the Atlas arrays. Semi-quantitative PCR analysis of BCL-6 expression in the populations of DCs studied here revealed the following pattern: GM-CSF cultured monocytes, immature DCs (G4) and IFN γ-treated DCs maintained a high level of BCL-6-specific mRNA. Dendritic cells maturation-induced with IL-7, TNF α or s CD40 LT, expressed significantly lower amounts of BCL-6 transcripts, similar to findings in the cDNA hybridization experiments. In separate experiments, using mature and immature DCs from other normal donors, the patterns of specific mRNA accumulation of BCL-6, CDS7, CD81 and CD53 were maintained (data not shown).

Discussion

As revealed by cluster analysis, the patterns of gene expression ‘in the populations of DCs, maturation-induced with either TNF α or s CD40 LT, were closely related. This finding is consistent with the structural similarity between these members of the TNF α gene super family, the sharing of many intermediates in their signaling pathways and regulation of transcription (Gruss, 1996). The pattern of cytokine gene expression in these mature DCs is also consistent with previous reports on the effects of TNF α and CD40L on immature DCs (Sallusto et al., 1999a). Dendritic cells cultured with s CD40 LT showed increased expression of chemokine genes active on memory and Th 2-type T cells (MDC, TARC, rants), as well as cytokines active on naive T cells (MIP3-β/ELC and IL-8) and Th 1-type T cells (MIG). In this same population of DCs, down-regulated expression of the genes for H-12 was found, and a series of pro-inflammatory cytokines such as MIF, NAP 2, IL-1 beta, IL-6 was also observed. The expression of the anti-inflammatory cytokine genes IL-10 and IL-1 RA was also reduced. A similar pattern of cytokine gene expression was observed in DCs maturation-induced with TNF α.

BCL6 is a transcriptional repressor of chemokine gene expression in murine macrophages (Toney et al., 2000). In BCL-6−/−knockout mice macrophages show increased expression of several Th2 type cytokines such as MCP-1 and MRP-1. Also demonstrated by the authors was the presence of BCL6 binding sites in the 5′ untranslated regions of the IL-8 and CD 23 genes. Both IL-8 (LYNAP) and CD 23 showed reciprocal expression with BCL6 in the maturation-induced DCs studied in the experiments (FIGS. 9, 11 and 13). Cluster analysis further showed that maturation-induced DCs (G4T and G4CD40L) had increased expression of helix-loop-helix id2, c-rel, IRF-4, HOX-5 and BTG-1 transcription factors. An observation of increased c-rel expression following maturation induction confirms a previous study of human dendritic cells (Neumann et al., 2000).

As expected, DCs treated with s CD40 LT showed increased cell surface expression of MHC class II, CD 80, CDS 6 and CD 83 proteins. Expression analysis showed increased levels of transcripts for CD 54 (ICAM-1), CD58 (LFA3), CD 86, CD 83, CD1 1a, CD 23. The level of mRNA specific for several molecules involved in cell-to-cell contact was reduced in DCs following CD 40 ligation. These molecules included CD 44H, CD 49B, CD 43, CD lie, CD18 and several transcripts encoding proteins with accessory functions, such as tsa-1/sca-1 (Saitoh et al., 1995), tetraspanins CD 9, 37, 53 and 81, and the inhibitory co-stimulatory molecule CD 153(CD30L) (Gattei et al., 1999), CXC3 fractalkine (Papadopoulos et al., 1999).

The differential expression of CD 37 and other tetraspanin molecules in immature and mature DCs is another novel finding. In the studies, abundant accumulation of mRNA for CD37 was detected in cultured monocytes and immature G7 DCs. Following maturation induction, however, the message for CD37 and four other tetraspanins is significantly reduced. CD 37 has been previously detected on mature B cells (Schwartz-Albiez et al., 1988) and has a putative role in T-cell-B-cell interactions (Knobeloch et al., 2000). CD37 is closely associated with MHC class II molecules and is selectively enriched (along with tetraspanins CD53 and CD81) in exosomes (Escola et al., 1998). Exosomes are formed when specialized endocytic vesicles containing processed antigen and MHC class II molecules (MIICs) fuse with the plasma membrane and are released in the extracellular space. Exosomes are able to prime T lymphocyte-dependent anti-tumor responses in vivo (Zitvogel et al., 1998). Following maturation, DCs are less able to present intact antigen to T cells (Sallusto & Lanzavecchia, 1994; Koch et al., 1995; Mellman et al. 1998). Down-regulation of genes coding for constituents of exosomes, such as CD 37, CD S3 and 81, may in part explain these observations.

Based on the expression profiles for several cytokines, lymphokines, transcription factors and cell surface molecules, the populations of mature DCs (induced with either s CD40L or TNF α) characterized in these studies share many characteristics with DCs polarized towards the DC2 type (Ria et al., 1998; Kapsenberg & Kalinski, 1999; Hashimoto et al., 2000). However, these populations also retained some characteristics of type 1 DCs. This conclusion is suggested by the detection in these DCs of transcripts specific for MlG, a chemokine active on Th 0- and Th 1-type T cells (Sallusto et al., 1998), MIP 3 β, a chemokine targeted to memory Th 1 (Randolph et al., 1999; Sallusto et al., 1999b), and IL-8 whose CXCR1 receptors are preferentially expressed on Th 1 cells (Bonecchi et al., 1998). Whether the mixed DC1-DC2 phenotype was as a result of heterogeneity in the DC populations or, alternatively, caused by incomplete maturation (Langenkamp et al., 2000) or polarization, could not be answered by the methods used. If the mixed DC1-DC2-type phenotype is common to other DC populations that have been used for adoptive transfer in humans (Dhodapkar et al., 1999), the successful induction of Th 1 and cytotoxic T-cell responses may have been driven by immature DC1-type subpopulation (Koch et al., 1995).

Comparison of expression data from G and G4 DCs with maturation-induced DCs (G4T and G4CD40L) showed reciprocal expression of many of the genes studied. Immature DCs showed increased expression of a cluster of genes formed by IL-12, IL-10, CX3C, IL-6 and IL-1 beta. In this cluster, maturation-induced DCs showed down-regulated expression of this same set of genes. Conversely, a second cluster of low expression, formed by IL-14, MIP-3 beta, MIG and TPO, was found in immature DCs. Following maturation, this group of genes was up-regulated. Similar to previous reports of gene expression in dendritic cells grown from CD14⁺-adherent monocytes (Hashimoto et al., 2000; Ishii et al., 2000), the studies showed that G4 DCs increased expression of the DC2-type chemokine genes (i.e. MDC, TARC and rants) relative to the monocyte population. If however, IL-12 production by DCs is the dominant force driving Th 1-type T-cell development (Kalinski et al., 1997b; Vieira et al., 2000), then the immature G4 DCs that still maintained detectable levels of IL-12 mRNA may be better able to initiate Th 1-type T-cell responses than the mature DCs populations that were characterized. Lastly, relative to the monocyte population (G), the expression of transcripts for the pro-survival Bcl-2 homologue Al (Lin et al., 1996) were decreased following culture with IL-4 or maturation induction. This observation is in accord with findings demonstrating that DCs maturation was accompanied by increased apoptotic susceptibility to HLA-DR-mediated apoptosis (Bertho et al., 2000).

Two populations of DCs, those cultured with IL-7 or induced by IFN γ, shared gene expression patterns with both mature and immature DCs examined in this study.

Overall, the gene expression pattern of GM-CSF cultured monocytes and immature DCs cultured in IL-7 or IL-4 were closely related. Both IL-4 and IL-7 receptors share the common receptor subunit responsible for mediating differentiation action of their ligands (He et al., 1995). By cluster analysis, immature DCs treated with IFN γ appeared to have a distinct pattern of gene expression relative to the other mature DCs studied. Again, this observation is consistent with the current understanding of the distinct signaling pathways used, by cells responding to INF γ. In the context of DC-based cancer immunotherapy, the immature dendritic cells grown in IL-7 or induced with IFN γ may represent populations that are more polarized towards the DC1 phenotype. For example, DCs grown in IL-7, despite expressing less MHC class II and co-stimulatory molecules, relative to mature DCs, are effective stimulators of cytotoxic T-cell and mixed-lymphocyte responses (Takahashi et al., 1997: Li et al., 2000). In addition, DCs grown in IL-7 show lower levels of transcription of DC2-type chemokines including MDC, TARC and the chemokine receptor CXCR5 (Legler et al., 1998; Ansel et al., 2000), while maintaining detectable IL-12 mRNA levels. Similarly, DCs induced with IFN γ have high cell surface levels of MHC class II molecules, CD 83 and CD 86. Relative to the other maturation-induced cells. DCs maturation-induced with IFN γ retain IL-12 mRNA expression. Dendritic cells, exposed to the Th 1-type lymphokine IFN γ, may themselves be more effective in priming Th 1 responses (Macatonia et al., 1995: Vieira et al., 2000) because they have not undergone the final maturational steps that lead to IL-12 transcript down-regulation (Ebner et al., 2001). The culture conditions used in these studies provide the basis for studying DCs in an immature or intermediate stage of development. In the context of DC-based immunotherapy, anti-tumor DC vaccines based on a single HLA class I (e.g., HLA-A2)-restricted peptide epitope appear promising but have serious shortcomings, such as providing a selective pressure for the escape of tumors that lose HLA-2 expression and are not applicable to HLA-A2-negative patients. One solution might be to return to the use of multiple epitope constructs or full-length antigenic proteins. Under this latter scenario, it is desirable that the DC population to maintain their antigen-processing functions. In this case it may be more effective to load antigen on DCs grown in IL-7 or maturation-induced with IFN γ for adoptive transfer. DC populations are being tested functionally to determine whether the hypotheses generated from these gene expression studies are valid.

The use of polystyrene beads in closed containers has a number of advantages over the use of open flasks for reproducibly generating dendritic cells, including sterility, risks of exposure for workers, higher yield, etc. Because of these factors, a closed system, e.g., flexible gas permeable plastic tissue culture bags, is preferred over the open flasks. The bags alone, however, do not provide an ideal surface for the attachment of DC precursor cells (monocytes). The introduction of selected polystyrene beads into the bags provides a surface that the monocytes easily adhere to. Once the monocytes have matured into DC, their adherence to the polystyrene surface, provided by the beads, is significantly reduced. At the end of the culture period, DCs no longer adhere to the beads and are harvested in the supernatant.

The beads are selected based, in part, on their size. Since more surface area is desirable, smaller beads in a larger quantity is preferred to larger beads in a smaller quantity.

Also, the specific gravity of the beads which allows them to settle after a period also contributes to their utility in the above-described methods. Since the monocytes adhered to the beads settle with the beads and thereby separate from the undesired cells (e.g., lymphocytes, platelets, etc.) which are removed by expressing off the supernatant.

Quality control is applied in the methods described above to comply with good manufacturing practices criteria.

While the application has been described with reference to specific embodiments, it should be understood that the description is not meant to be construed in a limiting sense, and the application is not limited to the precise embodiments described herein.

For example, while the closed system in the embodiments described above use a cell culture bag, other cell culture vessels may be used for the closed container. The container need not have any particular shape. It is preferable, but not essential, to have more than one port on the container to facilitate the transfer of materials in and out of the container. It is important, however, that the container is gas permeable to, for example, O₂ and CO₂. Also, it is important to maintain a ratio of (beads and container) surface area to container volume that allows the container to hold enough media to support the culture period, so that culture only needs to be fed once, rather than repeatedly.

Improvements and modifications which become apparent to persons of ordinary skill in the art after reading this disclosure, the drawings and the appended claims are deemed within the spirit and scope of the present application. It is therefore contemplated that the appended claims would cover any such modifications or improvements.

### REFERENCES

Ansel, K. M., Ngo, V. N., Hyman, P. L., Luther, S. A., Forster, R., Sedgwick, J. D., Browning, J. L., Lipp, M. & Cyster, J. G. (2000) “A chemokine-driven positive feedback loop organizes lymphoid follicles,” Nature, 406, 309-314.
Banchereau, J., Briere, F., Caux, C., Davoust, J., Lebecque, S., Liu, Y. J., Pulendran, B. & Palucka, K. (2000) Immunobiology of dendritic cells. Annual Review of Immunology, 18, 767-811.
Bartholeyns J., Romet-Lemonne J. L., Chokri M., Buyse M., Velu T., Bruyns C., Van de Winkel J. J., Heeney J., Koopman G., Malmsten M., De Groote D., Monsigny M., Midoux P., Alarcon B. (1998) 149 Cellular Vaccines. Res. Immunol. 647-49.
Bender, A., Sapp, M., Schuler, G., Steinman, R. M. & Bhardwaj, N. (1996) Improved methods for the generation of dendritic cells from nonproliferating progenitors in human blood. Journal of Immunological Methods, 196, 121-135.
Bernhard, H., Disis, M. L., Heimfeld, S., Hand, S., Gralow, J. R. & Cheever, M. A. (1995) Generation of immunostimulatory dendritic cells from human CD34+ hematopoietic progenitor cells of the bone marrow and peripheral blood. Cancer Research, 55, 1099-1104.
Bertho, N., Drenou, B., Laupeze, B., Berre, C. L., Amiot, L., Grosset, J. M., Fardel, 0., Charron, D., Mooney, N. & Fauchet, R. (2000) HLA-DR-mediated apoptosis susceptibility discriminates differentiation stages of dendritic/monocytic APC. Journal of Immunology, 164, 2379-2385.
Bonecchi, R., Bianchi, G., Bordignon, P. P., D'Ambrosio, D., Lang, R. Borsatti, A., Sozzani, S., Allavena, P., Gray, P, A, Mantovani, A. & Sinigaglia, F. (1998) Differential expression of chemokine receptors and chemotactic responsiveness of type 1 T helper cells (Th1s) and Th2s. Journal of Experimental Medicine, 187, 129-134.
Boyum, A. (1983) Isolation of human blood monocytes with Nycodenz, a pew non-ionic iodinated gradient medium. Scandinavian Journal of Immunology, 17, 429-436.
Brinckerhoff, L. H., Thompson, L. W. & Slingluff, Jr. C. L. (2000) Melanoma vaccines. Current Opinion in Oncology, 12, 163-173.
Caux, C., Massacrier, C., Vanbervliet, B., Dubois, B., Van Kooten, C., Durand, I. & Banchereau, J. (1994) Activation of human dendritic cells through CD40 cross-linking. Journal of Experimental Medicine, 180, 1263-1272.
Cella, M., Scheidegger, D., Palmer-Lehmann, K., Lane, P., Lanzavecchia, A. & Alber, G. (1996) Ligation of CD40 on dendritic cells triggers production of high levels of interleukin-12 and enhances Tcell stimulatory capacity: T-T help via APC activation. Journal of Experimental Medicine, 184, 747-752.
Clontech Laboratories (2001) Hemalology/Immunology Array Information (www document), URL http://atlasinfo.clon tech.com/atlasinfo/Atlaslnfo2/dynamicScripts/cDNAList. ssc?listClontechArrayUid=7&listClontechCatNo=7737-1.
Dhodapkar, M. V., Steinman, R. M., Sapp, M., Desai, H., Fossella, C. Krasovsky, J., Donahoe, S. M., Dunbar, P. R., Cerundolo, V., Nixon, D. F. & Bhardwaj, N. (1999) Rapid generation of broad T-cell immunity in humans after a single injection of mature dendritic cells. Journal of Clinical Investigation, 104, 173-180.
Dhodapkar, M. V., Krasovsky, J., Steinman, R. M. & Bhardwaj, N. (2000) Mature dendritic cells boost functionally superior CDS (+) T-cell in humans without foreign helper epitopes. Journal of Clinical Investigation, 105, R9-R14.
Di Nicola M., Anichini A., Mortarini R., Bregni M., Parmiani G., Gianni. A. M. (1998) Human dendritic cells: natural adjuvants in antitumor immunotherapy, 4 Cytokines Cell Mol. Ther. 265-73.
Ebner, S., Ratzinger, G., Krosbacher, B., Schmuth, M., Weiss, A., Reider, D., Kroczek, R. A., Herold, M., Heufler, C., Fritsch, P. & Romani, N. (2001) Production of IL-12 by human monocyte-derived dendritic cells is optimal when the stimulus is given at the onset of maturation, and is further enhanced by IL-4. Journal of Immunology, 166, 633-641, (1998).
Eisen, M. B., Spellman, P. T., Brown, P. O. & Botstein, D. (1998) Cluster analysis and display of genome-wide expression patterns. Proceedings of the National Academy of Sciences of the United States of America, 95, 14863-14868.
Escola, J. M., Kleijmeer, M. J., Stoorvogel, W, Griffith, J. M., Yoshie, 0. & Geuze, H. J. (1998) Selective enrichment of tetraspan proteins on the internal vesicles of multivesicular endosomes and on exosomes secreted by human B-lymphocytes. Journal of Biological Chemistry, 273, 20121-20127.
Ferrer, M., Yunta, M. & Lazo, P. A. (1998) Pattern of expression of tetraspanin antigen genes in Burkitt lymphoma cell lines. Clinical and Experimental Immunology, 113, 346-352.
Gattei, V., Degan, M., Rossi, P. M., de Iuliis, A., Mazzocco, F. T., Serraino, D., Zagonel, V, Aldinucci, D. & Pinto, A. (1999) CD30 ligand (CD30L)-expressing acute myeloid leukemias: a new model of paracrine interactions for the regulation of blast cells proliferation. Leukemia and Lymphoma, 35, 21-35.
Glaser, A., Zingsem, J., Zimmermann, R., Weisbach, V. & Eckstein, R. (1999) Collection of mononuclear cells in the Spectra for the generation of dendritic cells. Transfusion, 39, 661-662.
Granucci, F., Ferrero, E., Foti, M., Aggujaro, D., Vettoretto, K. & Ricciardi-Castagnoli, P. (1999) Early events in dendritic cell maturation induced by LPS. Microbes and Infection, 1, 1079-1084.
Grass, H. J. (1996) Molecular, structural, and biological characteristics of the tumor necrosis factor ligand superfamily. International Journal of Clinical and Laboratory Research, 26, 143-159.
Hartmann, G., Weiner, G. J. & Krieg, A. M. (1999) CpG DNA: a potent signal for growth, activation, and maturation of human dendritic cells. Proceedings of the National Academy of Sciences of the United States of America, 96, 9305-9310.
Hashimoto, S., Suzuki, T., Dong, H. Y., Nagai, S., Yamazaki, N. & Matsushima, K. (1999) Serial analysis of gene expression in human monocyte-derived dendritic cells. Blood, 94, 845-852.
Hashimolo, S. I., Suzuki, T., Nagai, S., Yamashita, T., Toyoda, N. & Matsushima, K. (2000) Identification of genes specifically expressed in human activated and mature dendritic cells through serial analysis of gene expression. Blood, 96, 2206-2214.
He, Y. W., Adkins, B., Furse, R. K. & Malek, T. R. (1995) Expression and function of the gamma c subunit of the FL-2, IL-4, and IL-7 receptors, Distinct interaction of gamma c in the IL-4 receptor. Journal of Immunology, 154, 1596-1605.
Ishii, M., Hashimoto, S., Tsutsumi, S., Wada, Y., Matsushima, K., Kodama, T. & Aburatani, H. (2000) Direct comparison of GeneChip and SAGE on the quantitative accuracy in transcript profiling analysis. Genomics, 68, 136-143.
Kalinski, P., Hilkens, C. M., Snijders, A., Snijdewint, F. G. & Kapsenberg, M. L. U997a) Dendritic cells, obtained from peripheral blood precursors in the presence of PGE2, promote Th2 responses. Advances in Experimental Medicine and Biology, 417, 363-367.
Kalinski, P., Hilkens, C. M., Snijders, A., Snijdewint, F. G. & Kapsenberg, M. L. (1997b) IL-12-deficient dendritic cells, generated in the presence of prostaglandin E2, promote type 2 cytokine production in maturing human naive T helper cells. Journal of Immunology, 159, 28-35.
Kalinski, P., Hilkens, C. M., Wierenga, E. A. & Kapsenberg, M. L. (1999) T-cell priming by type-1 and type-2 polarized dendritic cells: the concept of a third signal. Immunology Today, 20, 561-567.
Kapsenberg, M. L. & Kalinski, P. (1999) The concept of type 1 and type 2 antigen-presenting cells. Immunology Letters, 69, 5-6.
Knobeloch, K. P., Wright, M. D., Ochsenbein, A. F., Liesenfeld, 0., Lohler, J., Zinkernagel, R. M., Horak, I. & Orinska, Z. (2000) Targeted inactivation of the tetraspanin OD37 impairs T-cell-dependent B-cell response under suboptimal costimulatory conditions. Molecular and Cellular Biology, 20, 5363-5369.
Koch, E., Trockenbacher, B., Kampgen, E., Grauer, 0., Stossel, H., Livingstone, A. M., Schuler, G. & Romani, N. (1995) Antigen processing in populations of mature murine dendritic cells is caused by subsets of incompletely matured cells. Journal of Immunology, 155, 93-100.
Kodaira, Y., Nair, S. K., Wrenshall, L. E., Gilboa, E. & Platt, J. L. (2000) Phenotypic and functional maturation of dendritic cells mediated by heparan sulfate. Journal of Immunology, 165, 1599-1604.
Langenkamp, A., Messi, M., Lanzavecchia, A. & Sallusto, F. (2000) Kinetics of dendritic cell activation: impact on priming of TH1, TH2 and nonpolarized Tcells. Nature Immunology, 1, 311-316.
Lanzavecchia, A. (1999) Dendritic cell maturation and generation of immune responses. Haematologica, 84, 23-25.
Larsson, M., Messmer, D., Somersan, S., Fonteneau, J. F., Donahoe, S. M., Lee, M., Dunbar, P. R., Cerundolo, V. Julkunen, I., Nixon, D. F. & Bhardwaj, N. (2000) Requirement of mature dendritic cells for efficient activation of influenza A-specific memory CD8+ T cells. Journal of Immunology, 165, 1182-1190.
Legler, D. F., Loetscher, M., Roos, R. S., Clark-Lewis, I., Baggiolini, M. & Moser, B. (1998) B cell-attracting chemokine 1, a human CXC chemokine expressed in lymphoid tissues, selectively attracts B lymphocytes via BLR1/CXCR5. Journal of Experimental Medicine, 187, 655-660.
Li, L., Masucci, M. G. & Levitsky, V. (2000) Effect of interleukin-7 on the in vitro development and maturation of monocyte derived human dendritic cells. Scandinavian Journal of Immunology, 51, 361-371.
Lin, E. Y., Orlofsky, A., Wang, H. G., Reed, J. C. & Prystowsky, M. B. (1996) A1, a Bcl-2 family member, prolongs cell survival and permits myeloid differentiation. Blood, 87, 983-992.
Macatonia, S. E., Hosken, N. A., Litton, M., Vieira, P., Hsieh, C. S. Culpepper, J. A., Wysocka, M., Trinchieri, G., Murphy, K. M. & O'Garra, A. (1995) Dendritic cells produce IL-12 and direct the development of Th1 cells from naive CD4+ T cells. Journal of Immunology, 354, 5071-5079.
Maffei, A., Ayello, J., Skerrett, D., Papadopoulos, K., Harris, P. & Hesdorffer, C. (2000) A novel closed system utilizing styrene copolymer bead adherence for the production of human dendritic cells. Transfusion, 40, 1419-1420.
Mellman, I., Turley, S. J. & Steinman, R. M. (1998) Antigen processing for amateurs and professionals. Trends in Cell Biology, 8, 231-237.
Morse, M. A., Zhou, L. J., Tedder, T. F., Lyerly, H. K. & Smith, C. (1997) Generation of dendritic cells in vitro from peripheral blood mononuclear cells with granulocyte-macrophage-colony-stimulating factor, interleukin-4, and tumor necrosis factor-alpha for use in cancer immunotherapy. Annals of Surgery, 226, 6-16.
Neumann, M., Fries, H., Scheicher, C., Keikavoussi, P., Kolb-Maurer, A., Brocker, E., Serfling, E. & Kampgen, E. (2000) Differential expression of Rel/NF-kappaB and octamer factors is a hallmark of the generation and maturation of dendritic cells. Blood, 95, 277-285.
Papadopoulos, E. J., Sassetti, C., Saeki, H., Yamada, N., Kawamura, T., Fitzhugh, D. J., Saraf, M. A., Schall, T. Blauvelt, A., Rosen, S. D. & Hwang, S. T. (1999) Fractalkine, a CX3C chemokine, is expressed by dendritic cells and is up-regulated upon dendritic cell maturation. European Journal of Immunology, 29, 2551-2559.
Randolph, D. A., Huang, G., Carruthers, C. J., Bromley, L. E. & Chaplin, D. D. (1999) The role of CCR7 in TH1 and TH2 cell localization and delivery of B cell help in vivo. Science, 286, 2159-2162.
Rescigno, M., Granucci, F., Citterio, S., Foti, M. & Ricciardi-Castagnoli, P. (1999) Coordinated events during bacteria-induced DC maturation. Immunology Today, 20, 200-203.
Ria, F., Penna, G. & Adorini, L. U998) Th1 cells induce and Th2 inhibit antigen-dependent H-12 secretion by dendritic cells. European Journal of Immunology, 28, 2003-2016.
Rieser, C., Ramoner, R., Holtl, L., Rogatsch, H., Papesh, C., Stenzl, A., Bartsch, G. & Thurnher, M. (2000) Mature dendritic cells induce T-helper type-1-dominant immune responses in patients with metastatic renal cell carcinoma. Urologia Internationalis, 63, 151-159.
Romani, N. Reider, D., Heuer, M., Ebner, S., Kampgen, E., Eibl, B., Niederwieser, D. & Schuler, G. (1996) Generation of mature dendritic cells from human blood. An improved method with special regard to clinical applicability. Journal of Immunological Methods, 396, 137-151.
Saitoh, S., Kosugi, A., Noda, S., Yamamoto, N., Ogata, M., Minami, Y., Miyake, K. & Hamaoka, T. (1995) Modulation of TCR-mediated signaling pathway by thymic shared antigen-1 (TSA-1)/stem cell antigen-2 (Sca-2). Journal of Immunology, 155, 5574-5581.
Sallusto, F. & Lanzavecchia, A. (1994) Efficient presentation of soluble antigen by cultured human dendritic cells is maintained by granulocyte/macrophage colony-stimulating factor plus interleukin 4 and downregulated by tumor necrosis factor alpha. Journal of Experimental Medicine, 179, 1109-1118.
Sallusto, E., Cella, M., Danieli, C. & Lanzavecchia, A. (1995) Dendritic cells use macropinocytosis and the mannose receptor to, concentrate macromolecules in the major histocompatibility complex class II compartment: downregulation by cytokines and bacterial products. Journal of Experimental Medicine, 182, 389-400.
Sallusto, E., Schaerli, P., Loetscher, P., Schaniel, C., Lenig, D., Mackay, C. R., Qin, S. & Lanzavecchia, A. (1998) Rapid and coordinated switch in chemokine receptor expression during dendritic cell maturation. European Journal of Immunology, 28, 2760-2769.
Sallusto, E., Palermo, B., Lenig, D., Miettinen, M., Matikainen, S., Julkunen, I., Forster, R., Burgstahler, R., Lipp, M. & Lanzavecchia, A. (1999a) Distinct patterns and kinetics of chemokine production regulate dendritic cell function. European Journal of Immunology, 29, 1617-1625.
Sallusto, F., Lenig, D., Forster, R., Lipp, M. & Lanzavecchia, A. (1999b) Two subsets of memory T lymphocytes with distinct homing potentials and effector functions. Nature, 401, 708-712.
Schuler G., Steinman R. M. (1997) Dendritic cells as adjuvants for immune-mediated resistance to tumors, 186 J. Exp. Med. 1183-87.
Schwartz-Albiez, R., Dorken, B., Hofmann, W. & Moldenhauer, G. (1988) The B cell-associated CD37 antigen (gp40-52). Structure and subcellular expression of an extensively glycosylated glycoprotein. Journal of Immunology, 140, 905-914.
Siena, S., Di Nicola, M., Bregni, M., Mortarini, R., Anichini, A., Lombards, L., Ravagnani, E, Parmiani, G. & Gianni, A. M. (1995) Massive ex vivo generation of functional dendritic cells from mobilized CD34+ blood progenitors for anticancer therapy. Experimental Hematology, 23, 1463-1471.
Sparwasser, T., Koch, E. S., Vabulas, R. M., Heeg, K., Lipford, G. B. Ellwart, J. W. & Wagner, H. (1998) Bacterial DNA and immunostimulatory CpG oligonucleotides trigger maturation and activation of murine dendritic cells. European Journal of Immunology, 28, 2045-2054.
Takahashi, K., Honeyman, M. C. & Harrison, L. C. (1997) Dendritic cells generated from human blood in granulocyte macrophage-colony stimulating factor and interleukin-7. Human Immunology, 55, 103-116.
Tanaka, H., Demeure, C. E., Rubio, M., Delespesse, G. & Sarfati, M. (2000) Human monocyte-derived dendritic cells induce naive T cell differentiation into T helper cell type 2 (Th2) or Th1/Th2 effectors. Role of stimulator/responder ratio. Journal of Experimental Medicine, 192, 405-412.
Thomas, R. & Lipsky, P. E. (1994) Human peripheral blood dendritic cell subsets. Isolation and characterization of precursor and mature antigen-presenting cells. Journal of Immunology, 153, 4016-4028.
Thurner, B., Roder, C., Dieckmann, D., Heuer, M., Kruse, M., Glaser, A., Keikavoussi, P., Kampgen, E., Bender, A. & Schuler, G. (1999) Generation of large numbers of fully mature and stable dendritic cells from leukapheresis products for clinical application. Journal of Immunological Methods, 223, 1-15.
Tjoa, B. A. & Murphy, G. P. (2000) Development of dendritic-cell based prostate cancer vaccine. Immunology Letters, 74, 87-93.
Toney, L. M., Cattoretti, G., Graf, J. A., Merghoub, X, Pandolfi, P. P., Dalla-Favera, R., Ye, B. H. & Dent, A. L (2000) BCL-6 regulate chemokine gene transcription in macrophages. Nature Immunology, 3, 214-220.
Vieira, P. L., de Jong, E. C., Wierenga, E. A., Kapsenberg, M. L. & Kalinski, P. (2000) Development of Th1-inducing capacity in myeloid dendritic cells requires environmental instruction. Journal of Immunology, 164, 4507-4512.
Ye, Z., Gee, A. P., Bowers, W. E., Lamb, L. S., Turner, M. W. & Benslee Downey, P. J. (1996) In vitro expansion and characterization of dendritic cells derived from human bone marrow CD34+ cells. Bone Marrow Transplantation, 18, 997-1008.
Zhou, L. J. & Tedder, T. F. (1996) CD14+ blood monocytes can differentiate into functionally mature CD83+ dendritic cells. Proceedings of the National Academy of Sciences of the United States of America, 93, 2588-2592.
Zitvogel, L., Regnault, A., Lozier, A., Wolfers, J., Flament, C., Tenza, D., Ricciardi-Castagnoli, P., Raposo, G. & Amigorena, S. (1998) Eradication of established murine tumors using a novel cell-free vaccine: dendritic cell-derived exosomes. Nature Medicine, 4, 594-600.

What is claimed is:

1. A method of reproducibly generating dendritic cells, comprising the steps of:
(a) loading blood mononuclear cells into a cell culture container containing microcarrier beads therein;
(b) incubating for a predetermined time period tissue culture comprising the cells loaded in the container in step (a); and
(c) separating nonadherent cells and cells adhered to the beads.

2. A method of reproducibly generating dendritic cells, comprising the steps of:
(a) loading microcarrier beads into a cell culture container;
(b) loading blood mononuclear cells into the container;
(c) incubating for a predetermined time period tissue culture comprising the mononuclear cells loaded in the container in step (b); and
(d) separating nonadherent cells and cells adhered to the beads.

3. The method of claim 1, wherein the container comprises a gas permeable cell culture bag.

4. The method of claim 1, wherein the container is a closed vessel.

5. The method of claim 1, wherein the tissue culture incubated for the predetermined time period in step (b) is washed to remove nonadherent cells.

6. The method of claim 1, wherein after step (b) the beads are allowed to settle and supernatant is expressed off.

7. The method of claim 1 further comprising:
(d) preparing dendritic cell culture medium; and
(e) transferring the dendritic cell culture medium prepared in step (d) to the container after step (c).

8. The method of claim 7 further comprising:
(f) incubating the container for a second predetermined time period after step (e);
(g) agitating contents of the container incubated in step (f); and
(h) harvesting cell culture suspension by expression into transfer bags using a sterile connecting device after the beads agitated in step (g) are allowed to settle.

9. The method of claim 1, wherein after step (c) samples are removed from the container for quality control.

10. The method of claim 9, wherein the quality control includes at least one of viability staining, microbial analysis, cell enumeration, microscopic examination of dendritic cell morphology, and immunophenotyping to determine a purity of the dendritic cell preparation.

11. The method of claim 1, wherein the blood mononuclear cells are obtained by apheresis.

12. The method of claim 1, wherein a ratio of a combined surface area of the microcarrier beads and the container to a volume of the container volume is a value that allows the container to hold enough media for the predetermined time period of incubation in step (b).

13. The method of claim 1, wherein the microcarrier beads comprise styrene copolymer beads.

14. The method of claim 1, wherein the microcarrier beads comprise polystyrene copolymer beads.

15. An apparatus for reproducibly generating dendritic cells, comprising:
a cell culture container; and
a plurality of microcarrier beads contained within the cell culture container.

16. The apparatus of claim 15, wherein the container comprises a gas permeable cell culture bag.

17. The apparatus of claim 15, wherein the container is a closed vessel.

18. The apparatus of claim 15, wherein the microcarrier beads comprise styrene copolymer beads.

19. The apparatus of claim 15, wherein the microcarrier beads comprise polystyrene copolymer beads.

20. The apparatus of claim 15, wherein a ratio of a combined surface area of the microcarrier beads and the container to a volume of the container volume is a value that allows the container to hold enough media for a predetermined time period of incubation.
